SEQUENCE LISTING (62 sequences)

SEQ ID NO: 1 - DNA, 403 bp, Cannabis sativa
  source 1..403: mol_type = other DNA
  variation 202: allele = n = c/t
gctatattggctatattcaaacgttttgattttgaataggtactggtcccatgtggtgaattgccccagttgcagtgcagcatacaaagctctgaatgtggttaaagttgtgctcagtatcacctctcttgttttgattggtcttctgggtgtgaccaataagctagccactgcatcagttgctttgagagccacaatggtntcactctcaatactttgctttgcagcctcaaaatggctgaatcactttgtttacaagaactttcactttcatgactatattcatgcttttcattaattaacactacaaatatatattgtacatatataattcaatgtcatcagaagaaggaagagcttctgttattacacacaaacacacaattaagtataaaaagacagc

SEQ ID NO: 2 - DNA, 403 bp, Cannabis sativa
  source 1..403: mol_type = other DNA
  variation 202: allele = n = t/c
tttaaatatttcaagatgtgggcttcacatcttgaatacaatacaatggccaaagatgtttagtagctagcaaactgttatagggaccagaatgtatcaggtcatatccaagtttaaggctctcaaaagtgtcttcaaagagatgatcaatacagaaggatttctgatatccaagctgctaaatacatgctaaggagattcnggtgaaatgtcacaataaactacatcttgatcccatttctgggaaaattctcagggagatcaactataatgtactaacacttatccccaaaactaaatgccctaatacagtaagtgaatttaagcttatagcatgctgcaatttgatctacaaaatagctaccaaggtgatttgctcaaggctcatatacatactgcctaa

SEQ ID NO: 3 - DNA, 403 bp, Cannabis sativa
  source 1..403: mol_type = other DNA
  variation 202: allele = n = g/a
gaatgtctcttttcatattgtgctattggtgttgtccataagagaatttccctctctttgaagttttcagtgatttatatgcagaacctttgatcaggccatttgtgggttaagaggcatgttgtgatttttcaagtgtgtcattgttgaatacataaaagggtctccaaagcagcagctagctacttcaacacactttgtnacgattatgttgttggtctgtatttatggcactactactgatgaaacaaattcgaggtcaactcacttggtgtagcagttcattgagatttcgtgtggcttcagctacttcttggaatgccttggtatcaactcaaattcgagaattatatctcagtagaactatcaaacatttttctactagtgttgagagtgaaaagct

SEQ ID NO: 4 - DNA, 403 bp, Cannabis sativa
  source 1..403: mol_type = other DNA
  variation 202: allele = n = c/t
tcggaaagcaaaagcagctaggtcctcttacgctgctgcatcgatggtagttgatgctgttgaaaatgatggtaaatccacttgtgttcttggttccaggccaacttcgagccatgaagaatctagcaaaggaggttcggaatatgatagaccttttaaaatctctttagtgtcttatgaggagtatttaagagatcggaantttgtgaaactcgatactgaagcaaaggaagtgctaaataccatgttagctaagctgcagactcaagttgaattgttaacatctgaacgaggtattatccaaccaactgaggaaattttggtgaaattagtcaaggcatttctagaagcaggaaaggttaaagatttagccgagtttctcatcaaggtggagagagaagat

SEQ ID NO: 5 - DNA, 403 bp, Cannabis sativa
  source 1..403: mol_type = other DNA
  variation 202: allele = n = g/a
aaacagttcgtaaatgtatgtatttataggcaaggtcgggtggctcggtggacctgaattgttaagaacacgtcggatcaagttattattgaatgtctcagcattttcaagattggcatccggttctttggcaccaccgagccatggccaaccagcttcggtaacaacaacaggaatccccgaaaaatttgcagcctccatngcataataggtagcatccaccattgcatcaaacatgctactatagtggaaaagagtgttgggatcaacaatctgcttaaccgagggaagtggtcgaaaaatagcataatcaatcggaaaaatgccatccccttccgtgtatccataatatggatacgcatttaacatgtaataggaatttgtgtttttcaaaaactgaagg

SEQ ID NO: 6 - DNA, 403 bp, Cannabis sativa
  source 1..403: mol_type = other DNA
  variation 202: allele = n = g/a
agaggaagctagtgacgaagatgcacacttgacataaaccgaaatcaacgaattcaaaaccgaagtaacaaaacacaatccgaacttcacaaccgcgcaatgcatttgttgacactgcctttcatcatcaactacaagtccaaaagcactaagaacactagataaagtaaagtcatcaaccccaacactaaccctcctcatntcatgaaatgtttgaatagcagcaaagccatcattgttatgagaacaacaagtgatcatggcattgtagaaaacagtgtctctcatggtcaatggagtagcaaagaacacttcccttgccaacttaagattcccagctacagagtaagctgcaatcatagtcgttctagaaatgatatctggtttaggaatttggtcgaac

SEQ ID NO: 7 - DNA, 403 bp, Cannabis sativa
  source 1..403: mol_type = other DNA
  variation 202: allele = n = a/g
aagtaaataatatttacataagtgaattagaatgaagtaataagcaataaagtgccacaaactccaacaaatccaactacaaatcccactccaattcccatataaagccatgacatgttaagccacccttcatcatattcttcagcatcacttggatcatgaatatctttagggctatttggtgtctcatctccaagacatnaattgtttagtggaagtccgcacaatccatcattatcaatgtatatcgaagcattaaaactttgcaattgagtaccgataggaattcttccagacaatttgttacttgacaaattcaaaaaagatagagaagatatacttgccaagcttgttggaatgacactagaaagcttgttatgggataaatctagagaatccaact

SEQ ID NO: 8 - DNA, 403 bp, Cannabis sativa
  source 1..403: mol_type = other DNA
  variation 202: allele = n = a/g
agaggactaaaatacttgcatcaagattgtagtgcggcgattttacattttgacataaaacctcataacattcttctagattttgatttttctcctaaaatttctgacttcggccttgccaagttgtggcaaagggatgggagtggtgtatcactgttgaagggtagaggcacaattggatatacagcaccggagatgcatnacagaaattttggtgaagtatcttataaatctaatgtttatagttatggaatgatggttttagaaatggtgggcggaagaaaaaatattgatactagtgtttctcgaacaagtgcaatatattatccacattgggcacataagcatatcaatgatgatgatgatggtgagctcttgaaaaatatttgtgaagaaataatgg

SEQ ID NO: 9 - DNA, 403 bp, Cannabis sativa
  source 1..403: mol_type = other DNA
  variation 202: allele = n = t/c
tttatattacctacatgaaatcagacaacaacagcatatcgggggcttcactcatggagatggagacctactaccatttggttcatctgggtagcgatttggacttcggctaaggcttctctttgggcttcggcttctgcttcgactatggcttggacttctagctccacttggacccctagctgggcttcgacttgggctncttgatccattatatggtggagaacgagagtacgacctgtctcggctgtaccttctctcctgtggagacacagatctggcccgaagagggagtacgaaaattaatgtttaaacattgaaatatattaaatgcatgttttctcctattgctaaagatcccacatttttaatgctgactagagaagttgaaaagatatacttg

SEQ ID NO: 10 - DNA, 403 bp, Cannabis sativa
  source 1..403: mol_type = other DNA
  variation 202: allele = n = a/g
cgatcacttcgtagatgcatcctcccacaaggtagcacaattgtagaaagtgctaaatcatgctttattccatttgttctttttgtcttctctttttgcttaatcgaacgatgttgtgaacttgtagggttgtcaaattcgaagggagagcgcacatggagagagcgtttgttgcaacaatgtgagggccctgtttgatganctcccaactccacacctaattgtggagatcacaccattccctgaagggcctctcactgaaaaagattacaccaaagctgagaaattggagagggtacttagaactggcccgaacgtttgattcttctctcgagttaaatcatcgctgtctctcgttagaactacagcttaattgtatgtatgttttgagccttgtacatat

SEQ ID NO: 11 - DNA, 403 bp, Cannabis sativa
  source 1..403: mol_type = other DNA
  variation 202: allele = n = c/t
ttttatttctctcactttttttgcatactcttttctttccattcttctcgatcgtgctgaaatacctaataagacagtgacacagcatggcatgacacaattaatgggagcggttgcctttgcacaacaactcctcctcttccacctccactctgctgatcatatgggaccagagggacaatatcacttgctactccagctngtgattcttgtctctctggtcacatctctaatgggaatagggctaccgaagagtttcttagtgagttttgttaggtctcttagcattttgtttcaaggggtttggcttatggtgatggggtttatgttatggacaccatccttgatttccaaagggtgtttcatgcactatgaggaaggtcatcatgtggtgagatgctca

SEQ ID NO: 12 - DNA, 403 bp, Cannabis sativa
  source 1..403: mol_type = other DNA
  variation 202: allele = n = c/a
cacagcatctccattagaaggattaaacttatgagtactagcatgagcatacccacttgcaaacctaaaaagaccacttccaccaatcacaggcatttctctaaccttatcaaacacttggtttctaccaagaatggtgagagtgctcccattgtatttcccttgagttatatgaaaattcatagccataattagggcaatntcttcttgtgaagctaatccataaaacccttgagcttttcctagcaactttgagcttacttctggcccttctgtcaatggattgtcgatcatgctaaccgccccgaacccacttttcgatgcattggccggtggttggattattgccatcgcgctagggtttttgccgctgtatatgtcgtgccaatagaaccgaaagtgg

SEQ ID NO: 13 - DNA, 403 bp, Cannabis sativa
  source 1..403: mol_type = other DNA
  variation 202: allele = n = c/g
gctaacaagttaaacaccaagatacaaattctaccacatctttcttcatcctgcacaactagtagaagggtcttaaatcttgatacatcttggtagtgccaagatgcagtgagtatggagtggtatgagcttctttgaacacaaactctggttttatacaatagcatcccattacttgataccgtaaaatatttagtttgancagctaaaaccccttcccagatctttaccagttctggatcagttaactgagcagctttaattctgtctaaaaaaccagattgcaatgtcaaattatgaagtttgcctatgacaaactcaatgcctactctgaccgtatcatctgctagctgaggggaaatcttaatcatactagttacccgtccaggatcctttttgctca

SEQ ID NO: 14 - DNA, 403 bp, Cannabis sativa
  source 1..403: mol_type = other DNA
  variation 202: allele = n = a/g
acttatcttccttcctaagtccctgcctgccgctaacttttaccaaagccttaaaaaaaaattgttcaaagtcctgaaattccaaaactacccttacaaaaaaaaataatattcttttttcctcaaaaactacccttattattatttttttcctcaaaaacttgaaattacgcgggccaacccggatcctgacccgggtccnggtccgggtccggatccgacaacttccttcttcttcctcatttccaaaactttccgatgactattcgaatggacctcgctggaaaaagtcggactacaagcgggtcggtactcggggaaaagacgacccgacttgaaccgaaccccacaagcgttacaaagggttttgggaccgagcggaccggttcgccactgtggtgtc

SEQ ID NO: 15 - DNA, 20 bp, synthetic construct
  source 1..20: mol_type = other DNA
ccagttgcagtgcagcatac

SEQ ID NO: 16 - DNA, 24 bp, synthetic construct
  source 1..24: mol_type = other DNA
aagctcttccttcttctgatgaca

SEQ ID NO: 17 - DNA, 23 bp, synthetic construct
  source 1..23: mol_type = other DNA
agctcttccttcttctgatgaca

SEQ ID NO: 18 - DNA, 21 bp, synthetic construct
  source 1..21: mol_type = other DNA
aggctctcaaaagtgtcttca

SEQ ID NO: 19 - DNA, 21 bp, synthetic construct
  source 1..21: mol_type = other DNA
gccttgagcaaatcaccttgg

SEQ ID NO: 20 - DNA, 22 bp, synthetic construct
  source 1..22: mol_type = other DNA
aaggctctcaaaagtgtcttca

SEQ ID NO: 21 - DNA, 21 bp, synthetic construct
  source 1..21: mol_type = other DNA
ttgtgctattggtgttgtcca

SEQ ID NO: 22 - DNA, 20 bp, synthetic construct
  source 1..20: mol_type = other DNA
aagtagctgaagccacacga

SEQ ID NO: 23 - DNA, 20 bp, synthetic construct
  source 1..20: mol_type = other DNA
tgtgctattggtgttgtcca

SEQ ID NO: 24 - DNA, 20 bp, synthetic construct
  source 1..20: mol_type = other DNA
agtagctgaagccacacgaa

SEQ ID NO: 25 - DNA, 20 bp, synthetic construct
  source 1..20: mol_type = other DNA
gccaacttcgagccatgaag

SEQ ID NO: 26 - DNA, 21 bp, synthetic construct
  source 1..21: mol_type = other DNA
caccttgatgagaaactcggc

SEQ ID NO: 27 - DNA, 21 bp, synthetic construct
  source 1..21: mol_type = other DNA
accttgatgagaaactcggct

SEQ ID NO: 28 - DNA, 20 bp, synthetic construct
  source 1..20: mol_type = other DNA
gctcggtggacctgaattgt

SEQ ID NO: 29 - DNA, 20 bp, synthetic construct
  source 1..20: mol_type = other DNA
cacggaaggggatggcattt

SEQ ID NO: 30 - DNA, 20 bp, synthetic construct
  source 1..20: mol_type = other DNA
acggaaggggatggcatttt

SEQ ID NO: 31 - DNA, 20 bp, synthetic construct
  source 1..20: mol_type = other DNA
gaacttcacaaccgcgcaat

SEQ ID NO: 32 - DNA, 22 bp, synthetic construct
  source 1..22: mol_type = other DNA
agctgggaatcttaagttggca

SEQ ID NO: 33 - DNA, 21 bp, synthetic construct
  source 1..21: mol_type = other DNA
agttggcaagggaagtgttct

SEQ ID NO: 34 - DNA, 22 bp, synthetic construct
  source 1..22: mol_type = other DNA
acaaatcccactccaattccca

SEQ ID NO: 35 - DNA, 21 bp, synthetic construct
  source 1..21: mol_type = other DNA
gtcattccaacaagcttggca

SEQ ID NO: 36 - DNA, 20 bp, synthetic construct
  source 1..20: mol_type = other DNA
ttccaacaagcttggcaagt

SEQ ID NO: 37 - DNA, 20 bp, synthetic construct
  source 1..20: mol_type = other DNA
tcaagattgtagtgcggcga

SEQ ID NO: 38 - DNA, 20 bp, synthetic construct
  source 1..20: mol_type = other DNA
ttcttccgcccaccatttct

SEQ ID NO: 39 - DNA, 21 bp, synthetic construct
  source 1..21: mol_type = other DNA
catcaagattgtagtgcggcg

SEQ ID NO: 40 - DNA, 20 bp, synthetic construct
  source 1..20: mol_type = other DNA
caagtgcttgctgaggatgg

SEQ ID NO: 41 - DNA, 20 bp, synthetic construct
  source 1..20: mol_type = other DNA
gcgctacaggctctcagaat

SEQ ID NO: 42 - DNA, 20 bp, synthetic construct
  source 1..20: mol_type = other DNA
agtgcttgctgaggatggat

SEQ ID NO: 43 - DNA, 20 bp, synthetic construct
  source 1..20: mol_type = other DNA
gatgcatcctcccacaaggt

SEQ ID NO: 44 - DNA, 20 bp, synthetic construct
  source 1..20: mol_type = other DNA
cgggccagttctaagtaccc

SEQ ID NO: 45 - DNA, 20 bp, synthetic construct
  source 1..20: mol_type = other DNA
tcctcccacaaggtagcaca

SEQ ID NO: 46 - DNA, 20 bp, synthetic construct
  source 1..20: mol_type = other DNA
acgttcgggccagttctaag

SEQ ID NO: 47 - DNA, 20 bp, synthetic construct
  source 1..20: mol_type = other DNA
taatgggagcggttgccttt

SEQ ID NO: 48 - DNA, 21 bp, synthetic construct
  source 1..21: mol_type = other DNA
gcatctcaccacatgatgacc

SEQ ID NO: 49 - DNA, 20 bp, synthetic construct
  source 1..20: mol_type = other DNA
gacacagcatggcatgacac

SEQ ID NO: 50 - DNA, 21 bp, synthetic construct
  source 1..21: mol_type = other DNA
agtgcatgaaacaccctttgg

SEQ ID NO: 51 - DNA, 20 bp, synthetic construct
  source 1..20: mol_type = other DNA
gcatgagcatacccacttgc

SEQ ID NO: 52 - DNA, 20 bp, synthetic construct
  source 1..20: mol_type = other DNA
cggccaatgcatcgaaaagt

SEQ ID NO: 53 - DNA, 20 bp, synthetic construct
  source 1..20: mol_type = other DNA
acttccaccaatcacaggca

SEQ ID NO: 54 - DNA, 20 bp, synthetic construct
  source 1..20: mol_type = other DNA
caaaaaccctagcgcgatgg

SEQ ID NO: 55 - DNA, 20 bp, synthetic construct
  source 1..20: mol_type = other DNA
agtgccaagatgcagtgagt

SEQ ID NO: 56 - DNA, 20 bp, synthetic construct
  source 1..20: mol_type = other DNA
aggatcctggacgggtaact

SEQ ID NO: 57 - DNA, 21 bp, synthetic construct
  source 1..21: mol_type = other DNA
aggatcctggacgggtaacta

SEQ ID NO: 58 - DNA, 20 bp, synthetic construct
  source 1..20: mol_type = other DNA
gcctgccgctaacttttacc

SEQ ID NO: 59 - DNA, 20 bp, synthetic construct
  source 1..20: mol_type = other DNA
cttttccccgagtaccgacc

SEQ ID NO: 60 - DNA, 20 bp, synthetic construct
  source 1..20: mol_type = other DNA
cttcctaagtccctgcctgc

SEQ ID NO: 61 - DNA, 20 bp, synthetic construct
  source 1..20: mol_type = other DNA
cccgcttgtagtccgacttt

SEQ ID NO: 62 - protein, 551 aa, Cannabis sativa
MSNIQVLASSQLCDKIIARPTTKFHPSIWGDRFLHYNISEQDLVCKQERVEELIQVVKKEILSSNHDQLKLIDNLQRLGLSYHFESEIEKLLEQLSIGTHHQNNNIHDVSLRFRLLRQSGFKVSSSIFEKFKDEKGNFKESLITDVSGLLSLYEASHLSYVGESILDEALAFTTTHLKAIVANSKNHPLSHQISIALERPLRKTIERLHARFYISIYEKDASHNKLLLELAKLDFNVLQCFHKKELSEIMRWWKEHEFVKKFPFARDRMVELYFWILGVYYEPKYSRARKLLTKVIALTSITGDTYDAYGTIDKLELLTKAIQRWDINCLDKLEPEYLKTYYKVMLDSYEEFEKELKKEELYKLEYAKEEMKRTIGGYFEEARWLNEGYFPSFDEHLRVSYVSSANVLLIATSFVGMHDIVTHETLDWLSKHPNIVSASTLLSRFMDDIGSRKFEQERNHIPSTVDCYMKQYGVSEEEAIKELNKKVVTHWKEINEDFIRPTVVPFPILVRVLNFTKVIDLFYKEGDDQYTRVGKALKESIDALLIDSIPL

(19) United States
(12) Patent Application Publication
(10) Pub. No.: US 2026/0215382 A1
(43) Pub. Date: Jul. 30, 2026

(54) A QUANTITATIVE TRAIT LOCUS ASSOCIATED WITH SESQUITERPENE BIOSYNTHESIS IN CANNABIS

(71) Applicant: Puregene AG, Zeiningen (CH)

(72) Inventors: Claudio Cropano, Zeiningen (CH); Dániel Árpád Carrera, Zeiningen (CH); Gavin Mager George, Zeiningen (CH); Julian Koschmieder, Zeiningen (CH); Leron Katsir, Zeiningen (CH); Maximilian Moritz Vogt, Zeiningen (CH); Mercedes Thieme, Zeiningen (CH); Michael Edward Ruckle, Zeiningen (CH); Michele Wyler, Zeiningen (CH)

(21) Appl. No.: 19/146,897

(22) PCT Filed: Jan. 11, 2024
(86) PCT No.: PCT/IB2024/050267

§ 371 Date: Jul. 9, 2025

(30) Foreign Application Priority Data
Jan. 11, 2023 (GB) 2300435.1

(51) Int. Cl. A01H 1/04; A01H 1/02; A01H 5/12; A01H 6/28; C12Q 1/48; C12Q 1/686; C12Q 1/6869; C12Q 1/6895

(52) U.S. Cl. CPC A01H 1/045; A01H 1/021; A01H 5/12; A01H 6/28; C12Q 1/48; C12Q 1/686; C12Q 1/6869; C12Q 1/6895; C12Y 207/07; C12Q 2600/13; C12Q 2600/156

(57) ABSTRACT

Methods of identifying and characterizing a Cannabis spp. plant with respect to a distinct sesquiterpene trait comprising genotyping the plant for a quantitative trait locus (QTL) associated with a distinct sesquiterpene trait are provided. Also provided are methods of producing plants having a distinct sesquiterpene of interest based on defined allelic states of polymorphisms defining the QTL. Cannabis spp. plants having a distinct sesquiterpene trait of interest comprising defined allelic states of polymorphisms defining the QTL and characterized or produced by the methods described herein are further provided. The invention also relates to marker assisted selection and breeding methods for obtaining plants having a distinct sesquiterpene trait of interest and to a QTL associated with a distinct sesquiterpene trait. Plants having the QTL and extracts of such plants are also provided, as well as genes and causative polymorphisms responsible for the presence or absence of the distinct sesquiterpenes.

### BACKGROUND OF THE INVENTION

The invention relates to methods of identifying a Cannabis spp. plant comprising a quantitative trait locus (QTL) associated with a distinct sesquiterpene profile with reference, specifically, to α-eudesmol, β-eudesmol, epi-γ-eudesmol, and guaiol, and to Cannabis spp. plants having a distinct sesquiterpene trait. The invention also relates to marker assisted selection and marker assisted breeding methods for obtaining plants that have the distinct sesquiterpene trait defined by the presence or absence of α-eudesmol, β-eudesmol, epi-γ-eudesmol, and guaiol. The invention also provides methods of producing Cannabis spp. plants with the distinct sesquiterpene trait and plants produced by these methods, as well as plant extracts having altered sesquiterpene profiles, obtained from said plants.

Modern cannabis is the cross hybridization of three biotypes; Cannabis sativa L. ssp. indica, Cannabis sativa L. ssp. sativa, and Cannabis sativa L. ssp. ruderalis. Cannabis was divergently bred into two distinct, albeit tentative types, based on application. Hemp is primarily used for industrial purposes in feed, food, seed, fiber and oil production. Conversely, high-resin-type (HRT) cannabis is largely cultivated and bred for high concentrations of the pharmacological constituents, cannabinoids, derived from resin in the trichomes. However, recent interest from industrial producers in valuable, novel varieties calls for the convergence of these two types.

Cannabis produces phytocannabinoids a class of terpenoid that can act as antagonists and agonists of mammalian endocannabinoid receptors. The pharmacological action is derived from this ability of phytocannabinoids to disrupt and mimic endocannabinoids. Due to its psychoactive properties, one cannabinoid, delta-9-tetrahydrocannabinol (THC), the decarboxylation product of the plant-produced delta-9-tetrahydrocannabinolic acid (THCA), has received much attention in illegal or unregulated breeding programs, with modern HRT varieties having THC concentrations of 0.5% to 30%.

Glandular trichomes in Cannabis are also a major site of terpene biosynthesis-compounds that may act as plant defensive compounds and contribute to the aroma and medicinal qualities of cannabis. Terpenes have many commercial and industrial applications, including as flavour components in beer, as carriers for other small molecules, and as fragrances in cosmetics. Pharmacological effects of terpenes are an active area of research, with reported effects in humans including anxiolytic, antibacterial, anti-inflammatory, and sedative effects. In cannabis the interplay between terpenes and cannabinoids has been suggested to create an entourage effect, whereby the compounds enhance and modify each other's effect.

The biosynthetic pathways for terpene and cannabinoid biosynthesis share a common precursor, geranyl pyrophosphate (GPP), a product of the methylerythritol 4-phosphate (MEP) pathway localized to the plastid. GPP is the substrate for the large and diverse terpene synthase family responsible for the vast diversity of mono-terpenes. Sesquiterpenes are also found in cannabis, their biosynthesis depends on the biosynthesis of the precursor, farnesyl pyrophosphate (FPP), through an enzymatic pathway localized primarily to the peroxisome. Terpene synthase family enzymes are responsible for the biosynthesis of sesquiterpenes from FPP. Based on homologous terpene biosynthetic pathways in other organisms, many of the enzymes involved in GPP and FPP biosynthesis have been predicted. This is true for the enzymes for monoterpene and sesquiterpene biosynthesis as well. However, functional characterization of terpene synthase genes in cannabis, and in plants in general, is complicated by several limitations. In vitro characterization of terpene synthase genes may yield misleading results as they typically yield multiple products in varying amounts. The biosynthetic environment, substrate abundance, and specific protein cofactors may be essential for identifying the in vivo production and activity of terpene synthase genes. Genetic studies may be more powerful in identifying genes and specific polymorphisms associated with specific terpene profiles. For example, the in vivo biosynthetic pathway for many sesquiterpenes is unclear. In some determinations, cyclic sesquiterpenes are directly synthesized by a single terpene synthase, while other reports indicate many cyclic sesquiterpenes are synthesized from FPP through an intermediate step by a germacrene A synthase before being a substrate for an additional terpene synthase type protein.

The contribution of terpenes to Cannabis's character is poorly understood. Evidence suggests that terpenes may contribute to appealing or non-appealing Cannabis flavour profiles as well as enhancing or modulating the pharmacological effects of cannabinoids, the so-called entourage effect. In the global Cannabis market, terpenes are generally seen as adding value to cannabis flowers. One of the culturally identified effects of cannabis consumption is appetite stimulation post consumption. This characteristic of cannabis is pharmacologically relevant for encouraging appetite in patients, especially cancer patients undergoing chemotherapy. However, many consumers would prefer consuming cannabis without the aftereffect of increased hunger. Several reports indicate that cannabinoids may be involved in appetite modulation but the cyclic sesquiterpene, β-eudesmol, has also been implicated in stimulating appetite in mouse models. Interestingly, β-eudesmol may also suppress tumour growth by functioning as an anti-angiogenic compound.

Identifying and selecting for cannabis plants with distinct terpene composition is an emerging and important segment for the recreational and pharmacological Cannabis market. Terpene composition may also determine the amount of volatile organic compounds, potential irritants, released during cannabis combustion or vaporization. Distinct terpene profiles may also impact on a plant's resistance to pathogens and pests and give harvested flowers distinct post-harvest qualities. For example, the cyclic terpene guaiol has been reported to have insecticidal properties, targeting larval development.

In the present invention, cannabis populations with distinct terpene profiles of α-eudesmol, β-eudesmol, epi-γ-eudesmol, and guaiol are provided, together with polymorphisms identified as being associated with specific terpene profiles of these sesquiterpenes. Genetic markers for characterizing and identifying plants having specific sesquiterpene profiles with respect to α-eudesmol, β-eudesmol, epi-γ-eudesmol, and guaiol are further provided. The inventors have identified genes associated with these profiles and specific polymorphisms that regulate these genes, with the aim of developing varieties with the desired sesquiterpene profile.

### SUMMARY OF THE INVENTION

The present invention describes methods of identifying and/or characterizing a Cannabis spp. plant with respect to a distinct sesquiterpene trait comprising genotyping the plant for a quantitative trait locus (QTL) associated with a distinct sesquiterpene trait, and to methods of producing plants having a distinct sesquiterpene trait of interest based on defined allelic states of polymorphisms defining the QTL. Also described are Cannabis spp. plants having a distinct sesquiterpene trait of interest comprising defined allelic states of polymorphisms defining the QTL and plants identified, characterized, or produced by the methods described. The invention further relates to marker assisted selection and marker assisted breeding methods for obtaining plants having a distinct sesquiterpene trait of interest or for modulating the distinct sesquiterpene trait of cannabis plants, as well as to the distinct sesquiterpene QTL and genes and polymorphisms likely responsible for regulating the trait.

According to a first aspect of the present invention there is provided a method for characterizing a Cannabis spp. plant with respect to a distinct sesquiterpene trait, the method comprising the steps of: (i) genotyping at least one plant with respect to a distinct sesquiterpene QTL by detecting one or more polymorphisms associated with the distinct sesquiterpene trait as defined in any of Tables 2 to 5; and (ii) characterizing the one or more plants with respect to the distinct sesquiterpene QTL as having a sesquiterpene absence QTL, or a sesquiterpene presence QTL, based on the genotype at the polymorphism.

In a first embodiment of the method for characterizing a Cannabis spp. plant with respect to a distinct sesquiterpene trait, the polymorphism may be selected from the group consisting of “common_4518”, “common_4528”, and combinations thereof, as defined in any of Tables 2 to 5. These markers have all been validated for their predictive value for the distinct sesquiterpene QTL and trait.

In a second embodiment of the method for characterizing a Cannabis spp. plant with respect to a distinct sesquiterpene trait, the genotyping may be performed by any PCR-based detection method using molecular markers, by sequencing of PCR products containing the one or more polymorphisms, by targeted resequencing, by whole genome sequencing, or by restriction-based methods, for detecting the one or more polymorphisms.

According to a third embodiment of the method for characterizing a Cannabis spp. plant with respect to a distinct sesquiterpene trait, the molecular markers may be for detecting polymorphisms at regular intervals within the distinct sesquiterpene QTL such that recombination can be excluded. In an alternative embodiment, the molecular markers may be for detecting polymorphisms at regular intervals within the distinct sesquiterpene QTL such that recombination can be quantified to estimate linkage disequilibrium between a particular polymorphism and the distinct sesquiterpene phenotype. It will be appreciated by those of skill in the art that several possible markers may be designed for detecting the polymorphisms. For example, molecular markers may be for detecting polymorphisms such that recombination events can be detected to a resolution of 10′000 or 100′000 or 500′000 base pairs within the QTL. In one embodiment, the molecular markers may be designed based on a context sequence for the polymorphism as provided in Table 6 herein, or the molecular markers may be selected from the primer pairs as defined in Table 7.

In a fourth embodiment of the method for characterizing a Cannabis spp. plant with respect to a distinct sesquiterpene trait, the distinct sesquiterpene QTL is a quantitative trait locus having a sequence that corresponds to nucleotides 75059527-78081084 of NC_044377.1 of the CS10 genome and defined by one or more polymorphisms associated with the distinct sesquiterpene trait as defined in any of Tables 2 to 5. In another embodiment, the distinct sesquiterpene QTL may be defined by a genetic marker linked to the QTL.

According to a second aspect of the present invention, there is provided for a method of producing a Cannabis spp. plant having a distinct sesquiterpene trait of interest, the method comprising the steps of: (i) providing a donor parent plant having in its genome a distinct sesquiterpene QTL characterized by one or more polymorphisms associated with the distinct sesquiterpene trait of interest as defined in any of Tables 2 to 5; (ii) crossing the donor parent plant having the distinct sesquiterpene QTL with at least one recipient parent plant to obtain a progeny population of Cannabis spp. plants; (iii) screening the progeny population of Cannabis spp. plants for the presence of the distinct sesquiterpene QTL; and (iv) selecting one or more progeny plants having the distinct sesquiterpene QTL, wherein the mature plant displays the distinct sesquiterpene trait of interest. The distinct sesquiterpene trait of interest may be a sesquiterpene absence trait, or a sesquiterpene presence trait. In this way, the trait can be selected for in a plant using the distinct sesquiterpene QTL and markers therefor described herein.

In a first embodiment of the method of producing a Cannabis spp. plant having a distinct sesquiterpene trait of interest, the method may further comprise the steps of: (v) crossing the one or more progeny plants with the donor recipient plant; or (vi) selfing the one or more progeny plants.

According to a second embodiment of the method of producing a Cannabis spp. plant having a distinct sesquiterpene trait of interest, the screening may comprise genotyping at least one plant from the progeny population with respect to the distinct sesquiterpene QTL by detecting one or more polymorphisms associated with the distinct sesquiterpene trait of interest as defined in any of Tables 2 to 5.

In a third embodiment of the method of producing a Cannabis spp. plant having a distinct sesquiterpene trait of interest, the method may comprise a step of genotyping the donor parent plant with respect to the distinct sesquiterpene QTL by detecting one or more polymorphisms associated with the distinct sesquiterpene trait of interest as defined in any of Tables 2 to 5, preferably prior to step (i).

According to a fourth embodiment of the method of producing a Cannabis spp. plant having a distinct sesquiterpene trait of interest, the genotyping may be performed by a PCR-based detection method using molecular markers, by sequencing of PCR products containing the one or more polymorphisms, by targeted resequencing, by whole genome sequencing, or by restriction-based methods, for detecting the one or more polymorphisms.

In a fifth embodiment of the method of producing a Cannabis spp. plant having a distinct sesquiterpene trait of interest, the molecular markers may be for detecting polymorphisms at regular intervals within the distinct sesquiterpene QTL such that recombination can be excluded. In an alternative embodiment, the molecular markers may be for detecting polymorphisms at regular intervals within the distinct sesquiterpene QTL such that recombination can be quantified to estimate linkage disequilibrium between a particular polymorphism and the distinct sesquiterpene trait of interest. For example, molecular markers may be for detecting polymorphisms such that recombination events can be detected to a resolution of 10′000 or 100′000 or 500′000 base pairs within the QTL. It will be appreciated by those of skill in the art that several possible markers may be designed for detecting the polymorphisms. In one embodiment, the molecular markers may be designed based on a context sequence for the polymorphism described in Table 6 or may be selected from the primer pairs defined in Table 7.

According to a further embodiment of the method of producing a Cannabis spp. plant having a distinct sesquiterpene trait of interest, the distinct sesquiterpene QTL is a sesquiterpene absence QTL, or a sesquiterpene presence QTL defined by the allelic state of the polymorphisms as provided in any of Tables 2 to 5. In one embodiment, the distinct sesquiterpene trait of interest is a sesquiterpene absence trait, and the distinct sesquiterpene QTL is a sesquiterpene absence QTL. Of particular use in producing a Cannabis spp. plant having a distinct sesquiterpene trait of interest, are the polymorphisms selected from the group consisting of “common_4518”, “common_4528”, and combinations thereof, as defined in any of Tables 2 to 5, which have been validated for their predictive value for the distinct sesquiterpene QTL and trait, and particularly to a combination of these polymorphisms.

According to a third aspect of the present invention there is provided for a method of producing a Cannabis spp. plant that has a distinct sesquiterpene trait of interest, the method comprising introducing a distinct sesquiterpene QTL characterized by one or more polymorphisms associated with the distinct sesquiterpene trait of interest as defined in any of Tables 2 to 5 into a Cannabis spp. plant, wherein said distinct sesquiterpene QTL is associated with the distinct sesquiterpene trait of interest in the plant. In one embodiment, introducing the distinct sesquiterpene QTL comprises crossing a donor parent plant having the distinct sesquiterpene QTL characterized by one or more polymorphisms associated with the distinct sesquiterpene trait of interest with a recipient parent plant. In an alternative embodiment, introducing the distinct sesquiterpene QTL characterized by one or more polymorphisms associated with the distinct sesquiterpene trait of interest comprises genetically modifying the Cannabis spp. plant. Several methods of genetic modification are known to those of skill in the art, including targeted mutagenesis, genome editing, and gene transfer. For example, a distinct sesquiterpene QTL comprising one or more of the polymorphisms associated with the distinct sesquiterpene trait of interest as defined in any of Tables 2 to 5 herein may be introduced into a plant by mutagenesis and/or gene editing. In particular, the methods of genetically modifying a plant may be selected from the group consisting of CRISPR-Cas9 targeted gene editing, heterologous gene expression using various expression cassettes, TILLING, and non-targeted chemical mutagenesis using e.g., EMS. For example, CRISPR-Cas9 targeted gene editing may be achieved using a guide RNA. Alternatively, a cannabis spp. plant may be transformed with a cassette containing the distinct sesquiterpene QTL associated with the distinct sesquiterpene trait of interest or a part thereof, via any transformation method known in the art.

In one embodiment of the method of producing a Cannabis spp. plant that has a distinct sesquiterpene trait of interest, the distinct sesquiterpene QTL is a quantitative trait locus having a sequence that corresponds to nucleotides 75059527-78081084 of NC_044377.1 with reference to the CS10 genome and defined by one or more polymorphisms associated with the distinct sesquiterpene trait as defined in any of Tables 2 to 5. In another embodiment, the distinct sesquiterpene QTL may be defined by a genetic marker linked to the distinct sesquiterpene QTL.

According to a fourth aspect of the present invention there is provided for a Cannabis spp. plant characterized according to the method for characterizing a Cannabis spp. plant with respect to a distinct sesquiterpene trait as described herein. In some embodiments, the Cannabis spp. plant characterized according to the method of characterizing a Cannabis spp. plant having a distinct sesquiterpene trait of interest as described herein is not exclusively obtained by means of an essentially biological process.

In a fifth aspect of the present invention there is provided for a Cannabis spp. plant produced according to the method of producing a Cannabis spp. plant having a distinct sesquiterpene trait of interest as described herein. In some embodiments, the Cannabis spp. plant produced according to the method of producing a Cannabis spp. plant having a distinct sesquiterpene trait of interest as described herein is not exclusively obtained by means of an essentially biological process.

According to a further aspect of the present invention there is provided for a Cannabis spp. plant comprising a distinct sesquiterpene QTL characterized by one or more polymorphisms associated with a distinct sesquiterpene trait of interest as defined in any of Tables 2 to 5. In some embodiments, the plant is not exclusively obtained by means of an essentially biological process.

According to another aspect of the present invention there is provided for a quantitative trait locus that controls a distinct sesquiterpene trait in Cannabis spp., wherein the quantitative trait locus has a sequence that corresponds to nucleotides 75059527-78081084 of NC_044377.1 of the CS10 genome and defined by one or more polymorphisms associated with the distinct sesquiterpene trait as defined in any of Tables 2 to 5, or a genetic marker linked to the QTL. In some embodiments, the quantitative trait locus may be provided as an isolated nucleic acid molecule.

According to yet a further aspect of the present invention there is provided for a Cannabis spp. plant comprising the quantitative trait locus or a nucleic acid comprising the QTL as defined herein.

In yet a further aspect of the present invention, there is provided for a plant extract obtainable from a Cannabis spp. plant described herein.

According to another aspect of the present invention, there is provided for an isolated gene that controls a distinct sesquiterpene trait in a Cannabis spp. plant, wherein the gene is selected from the group consisting of LOC115695864 encoding a protein with homology to a germacrene D synthase, LOC115695865 encoding a terpene synthase, and LOC115695866 encoding a terpene synthase, with reference to Table 8 herein. In one embodiment, the gene is LOC115695864 encoding a protein with homology to a germacrene D synthase. According to a further embodiment, the gene is LOC115695864 encoding a protein with homology to a germacrene D synthase (SEQ ID NO:62) and, optionally comprising a single nucleotide polymorphism that results in an amino acid substitution at position 147 and/or position 303 of the protein, with reference to SEQ ID NO:62. Preferably, the single nucleotide polymorphism results in an amino acid substation of 147S>P and/or 303G>D with reference to SEQ ID NO:62. In some embodiments, the gene may be provided as an isolated nucleic acid molecule.

### BRIEF DESCRIPTION OF THE FIGURES

Non-limiting embodiments of the invention will now be described by way of example only and with reference to the following figures:

FIG. 1: Graphs depicting the presence or absence of guaiol, α-eudesmol, β-eudesmol, epi-γ-eudesmol in a subset of the F2 populations tested. The F2 population designation is shown below each plot while the specific sesquiterpene is given above. The Y-axis shows plant count—solid bars indicate presence of the compounds, while dashed bars indicate absence of the compounds.

FIG. 2: A correlation plot of the presence or absence of guaiol, «-eudesmol, β-eudesmol, epi-γ-eudesmol in the combined Cannabis F2 population used. The correlation coefficient is given for each correlation.

FIG. 3: Structures of guaiol, α-eudesmol, β-eudesmol, epi-γ-eudesmol are shown, taken from PubChem.

FIG. 4: Amino acid sequence of a germacrene D synthase homolog in Cannabis sativa encoded by the LOC115695864 gene with protein ID XP_030478815.1 according to NCBI.

### SEQUENCES

The nucleic acid and amino acid sequences listed herein and in any accompanying sequence listing are shown using standard letter abbreviations for nucleotide bases, and the standard one- or three-letter abbreviations for amino acids. It will be understood by those of skill in the art that only one strand of each nucleic acid sequence is shown, but that the complementary strand is included by any reference to the displayed strand.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims, which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items. It is, however, contemplated as a specific embodiment of the present disclosure that the term “comprising” encompasses the possibility of no further members being present, i.e., for the purpose of such an embodiment “comprising” is to be understood as having the meaning of “consisting of”.

In investigating cannabis plants from an F2 sub-population the inventors detected a surprising and distinct sesquiterpene profile, specifically defined by the presence or absence of α-eudesmol, β-eudesmol, epi-γ-eudesmol, and guaiol. This phenomenon was further investigated, and several polymorphisms associated with this trait were identified. The inventors further identified genetic markers to identify cannabis plants with the distinct sesquiterpene phenotype, providing a method to select for the presence or absence of the distinct sesquiterpene profile defined by the presence or absence of α-eudesmol, β-eudesmol, epi-γ-eudesmol, and guaiol.

Methods are provided herein for characterizing, identifying and obtaining plants having a distinct sesquiterpene trait of interest, using a molecular marker detection technique. The inventors of the present invention have further produced and selected for the distinct sesquiterpene trait in Cannabis spp. plants by crossing plants with the distinct sesquiterpene trait with plants that do not display the distinct sesquiterpene trait. Also demonstrated herein, the inventors were able to use genome wide association (GWA) to identify single nucleotide polymorphisms (SNPs) associated with the distinct sesquiterpene trait; these SNPs were verified as genetic markers for identifying plants carrying the sesquiterpene trait of interest. The inventors used the methods described herein to identify candidate genes that are causative for the distinct sesquiterpene trait. This finding provides for the improvement of methods for producing plants displaying various sesquiterpene traits and modulating the sesquiterpene profiles of Cannabis spp. plants.

Tables 2 to 5 herein provide several SNPs which define the QTL associated with the distinct sesquiterpene trait. In some embodiments one or more of the identified SNPs can be used to incorporate the distinct sesquiterpene trait of interest from a donor plant, containing the QTL associated with the trait, into a recipient plant. For example, the incorporation of the distinct sesquiterpene trait of interest may be performed by crossing a donor parent plant to a recipient parent plant to produce plants containing a haploid genome from both parents. Recombination of these genomes provides F1 progeny where each haploid complement of chromosomes, of the diploid genome, is comprised of genetic material from both parents.

In some embodiments, methods of identifying the QTL that is characterized by a haplotype comprising a series of polymorphisms in linkage disequilibrium are provided. The QTL displays limited frequency of recombination within the QTL. Preferably the polymorphisms are selected from any one provided in Tables 2 to 5 herein, representing the distinct sesquiterpene QTL. Molecular markers may be designed for use in detecting the presence of the polymorphisms and thus the QTL. Further, the identified QTL and the associated molecular markers may be used in a cannabis breeding program to predict the distinct sesquiterpene trait of interest of plants in a breeding population and can be used to produce cannabis plants that display a distinct sesquiterpene trait of interest, compared to the plants from which they are derived. The QTL identified herein, and the markers associated with the QTL, can be used to modulate the sesquiterpene profile in Cannabis spp. plants.

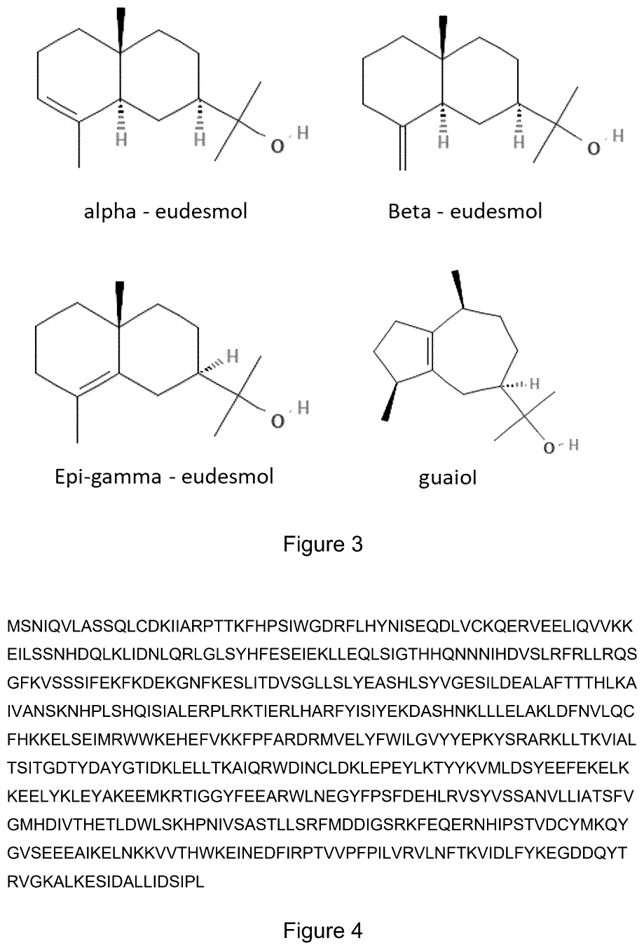

As used herein, reference to a plant's or a variety's “sesquiterpene profile” or a plant or variety with a “distinct sesquiterpene trait” refers to a plant or variety characterized by the presence or absence of the sesquiterpenes: α-eudesmol, β-eudesmol, epi-γ-eudesmol, and guaiol, conferred by the distinct sesquiterpene QTL identified herein. The IUPAC names of these sesquiterpene compounds are provided as: α-eudesmol: 2-[(2R,4aR,8aR)-4a,8-dimethyl-2,3,4,5,6,8a-hexahydro-1H-naphthalen-2-yl]propan-2-ol; β-eudesmol: 2-[(2R,4aR,8aS)-4a-methyl-8-methylidene-1,2,3,4,5,6,7,8a-octahydronaphthalen-2-yl]propan-2-ol; epi-γ-eudesmol: 2-[(2R,4aR)-4a,8-dimethyl-2,3,4,5,6,7-hexahydro-1H-naphthalen-2-yl]propan-2-ol; and guaiol: 2-[(3S,5R,8S)-3,8-dimethyl-1,2,3,4,5,6,7,8-octahydroazulen-5-yl]propan-2-ol. FIG. 3 herein depicts the molecular structures of these compounds. The content of sesquiterpenes is calculated in % of the dry mass of cannabis flower (% w/w) at the time of harvest.

A “distinct sesquiterpene trait of interest” refers to the state of the plant with respect to the distinct sesquiterpene trait and includes the sesquiterpene absence trait and sesquiterpene presence trait.

A “sesquiterpene absence trait” is defined by the absence of α-eudesmol, β-eudesmol, epi-γ-eudesmol, and/or guaiol.

A “sesquiterpene presence trait” is defined by the presence of α-eudesmol, β-eudesmol, epi-γ-eudesmol, and/or guaiol.

As used herein, “absence” with respect to sesquiterpene content is defined as an amount that is undetectable using the methods provided herein to determine sesquiterpene content.

As used herein, “presence” with respect to sesquiterpene content is defined as an amount that is detectable, regardless of amount, using the methods provided herein to determine sesquiterpene content.

The “time of harvest” is defined with respect to the maturity of the flower, where approximately greater than 50% of the pistils have turned brown in appearance. Alternatively, the time of harvest can also be determined by initiation of flowering for hemp-type cannabis or by other agronomic criteria common in the art.

It is a particular aim of the present invention to identify and characterize a plant for the distinct sesquiterpene trait of interest early in the plant lifecycle, particularly prior to the plant displaying the distinct sesquiterpene trait of interest, or to introduce a distinct sesquiterpene trait of interest into a breeding population of plants early on in a breeding program. This can be achieved by genotyping the plant using molecular markers for detecting the QTL associated with the distinct sesquiterpene trait prior to the time of harvest.

As used herein a “quantitative trait locus” or “QTL” is a polymorphic genetic locus with at least two alleles that differentially affect the expression of a continuously varying phenotypic trait when present in a plant or organism which is characterized by a series of polymorphisms in linkage disequilibrium with each other.

As used herein, the term “distinct sesquiterpene QTL” or “distinct sesquiterpene quantitative trait locus” refers to a quantitative trait locus characterized by one or more polymorphisms having an allelic state associated with the distinct sesquiterpene trait of interest, as described in Tables 2 to 5, or characterized by combinations of such polymorphisms.

In some cases, it is desirable to obtain a plant displaying a sesquiterpene absence trait, for example to reduce appetite stimulation or to obtain a specific flavour profile or to reduce volatile organic compounds released upon combustion or vaporization. In other embodiments, it is desirable to obtain a plant displaying a sesquiterpene presence trait, for example to obtain an alternative flavour profile. It may also be advantageous from a pharmaceutical perspective to have a plant displaying the sesquiterpene presence trait, for example for enhancement or modulation of the pharmacological effects of cannabinoids, to stimulate appetite, or even suppress tumour growth. A further possible advantage to having a plant displaying a distinct sesquiterpene trait of interest is to impart resistance to pathogens and pests and give harvested flowers distinct post-harvest qualities. Thus, depending on the application, it is an objective of the invention to provide for cannabis plants having a sesquiterpene absence trait or a sesquiterpene presence trait by genotyping and/or selecting plants for a sesquiterpene absence QTL or sesquiterpene presence QTL as described herein.

As used herein, “sesquiterpene absence QTL” or “sesquiterpene absence quantitative trait locus” refers to a quantitative trait locus characterized by one or more polymorphisms having an allelic state associated with the sesquiterpene absence trait, as described in Tables 2 to 5.

As used herein, “sesquiterpene presence QTL” or “sesquiterpene presence quantitative trait locus” refers to a quantitative trait locus characterized by one or more polymorphisms having an allelic state associated with the sesquiterpene presence trait, as described in Tables 2 to 5.

As described herein, in one embodiment it is desirable to obtain a plant displaying a distinct sesquiterpene haplotype.

As used herein, “haplotypes” refer to patterns or clusters of alleles or single nucleotide polymorphisms that are in linkage disequilibrium and therefore inherited together from a single parent. The term “linkage disequilibrium” refers to a non-random segregation of genetic loci or markers. Markers or genetic loci that show linkage disequilibrium are considered linked.

As used herein, the term “distinct sesquiterpene haplotype” refers to the subset of the polymorphisms contained within the distinct sesquiterpene QTL which exist on a single haploid genome complement of the diploid genome, and which are in linkage disequilibrium with the distinct sesquiterpene trait.

As used herein, the term “donor parent plant” refers to a plant having a distinct sesquiterpene haplotype or one or more distinct sesquiterpene alleles associated with the distinct sesquiterpene trait of interest.

As used herein, the term “recipient parent plant” refers to a plant having a distinct sesquiterpene haplotype or one or more distinct sesquiterpene alleles not associated with the distinct sesquiterpene trait of interest.

The term “distinct sesquiterpene allele” refers to the haplotype allele within a particular QTL that confers, or contributes to, the distinct sesquiterpene trait of interest, or alternatively, is an allele that allows the identification of plants with the distinct sesquiterpene trait of interest, that can be included in a breeding program (“marker assisted breeding”, “marker assisted selection”, or “genomic selection”).

The term “crossed” or “cross” means the fusion of gametes via pollination to produce progeny (e.g., cells, seeds, or plants). The term encompasses both sexual crosses (the pollination of one plant by another) and selfing (self-pollination, e.g., when the pollen and ovule are from the same, or genetically identical plant). The term “crossing” refers to the act of fusing gametes via pollination to produce progeny.

The term “GWAS” or “Genome wide association study” or “GWA” or “Genome wide association” as used herein refers to an observational study of a genome-wide set of genetic variants or polymorphisms in different individual plants to determine if any variant or polymorphism is associated with a trait, specifically the distinct sesquiterpene trait.

As used herein a “polymorphism” is a particular type of variance that includes both natural and/or induced multiple or single nucleotide changes, short insertions, or deletions in a target nucleic acid sequence at a particular locus as compared to a related nucleic acid sequence. These variations include, but are not limited to, single nucleotide polymorphisms (SNPs), indel/s, genomic rearrangements, and gene duplications.

As used herein, the term “LOD score” or “logarithm (base 10) of odds” refers to a statistical estimate used in linkage analysis, wherein the score compares the likelihood of obtaining the test data if the two loci are indeed linked, to the likelihood of observing the same data purely by chance. The LOD score is a statistical estimate of whether two genetic loci are physically near enough to each other (or “linked”) on a particular chromosome that they are likely to be inherited together. A LOD score of 3 or higher is generally understood to mean that two genes are located close to each other on the chromosome. In terms of significance, a LOD score of 3 means the odds are 1,000:1 that the two genes are linked and therefore inherited together.

As used herein, the term “quantile-quantile” or “Q-Q” refers to a graphical method for comparing two probability distributions by plotting their quantiles against each other. If the two distributions being compared are similar, the points in the Q-Q plot will approximately lie on the line y=x. If the distributions are linearly related, the points in the Q-Q plot will approximately lie on a line, but not necessarily on the line y=x. Q-Q plots can also be used as a graphical means of estimating parameters in a location-scale family of distributions.

As used herein, a “causal gene” is the specific gene having a genetic variant (the “causal variant”) which is responsible for the association signal at a locus and has a direct biological effect on the distinct sesquiterpene trait. In the context of association studies, the genetic variants which are responsible for the association signal at a locus are referred to as the “causal variants”. Causal variants may comprise one or more “causal polymorphisms” that have a biological effect on the phenotype.

The term “nucleic acid” encompasses both ribonucleotides (RNA) and deoxyribonucleotides (DNA), including cDNA, genomic DNA, isolated DNA and synthetic DNA. The nucleic acid may be double-stranded or single-stranded. Where the nucleic acid is single-stranded, the nucleic acid may be the sense strand or the antisense strand. A “nucleic acid molecule” or “polynucleotide” refers to any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides. By “cDNA” is meant a complementary or copy DNA produced from an RNA template by the action of RNA-dependent DNA polymerase (reverse transcriptase).

In some embodiments, the nucleic acid molecules of the invention may be operably linked to other sequences. By “operably linked” is meant that the nucleic acid molecules, such as those comprising the QTL of the invention or gene(s) identified herein, and regulatory sequences are connected in such a way as to permit expression of the proteins when the appropriate molecules are bound to the regulatory sequences. Such operably linked sequences may be contained in vectors or expression constructs which can be transformed or transfected into plant cells or plants for expression. A “regulatory sequence” refers to a nucleotide sequence located either upstream, downstream or within a coding sequence. Generally regulatory sequences influence the transcription, RNA processing or stability, or translation of an associated coding sequence. Regulatory sequences include but are not limited to: effector binding sites, enhancers, introns, polyadenylation recognition sequences, promoters, RNA processing sites, stem-loop structures, translation leader sequences and the like.

The term “promoter” refers to a DNA sequence that is capable of controlling the expression of a nucleic acid coding sequence or functional RNA. A promoter may be based entirely on a native gene, or it may be comprised of different elements from different promoters found in nature. Different promoters are capable of directing the expression of a gene at different stages of development, or in response to different environmental or physiological conditions. An “inducible promoter” is promoter that is active in response to a specific stimulus. Several such inducible promoters are known in the art, for example, chemical inducible promoters, developmental stage inducible promoters, tissue type specific inducible promoters, hormone inducible promoters, environment responsive inducible promoters.

The term “isolated”, as used herein means having been removed from its natural environment. Specifically, the nucleic acid(s) or gene(s) identified herein may be isolated nucleic acid(s) or gene(s), which have been removed from plant material where they naturally occur.

The term “purified”, relates to the isolation of a molecule or compound in a form that is substantially free of contamination or contaminants. Contaminants are normally associated with the molecule or compound in a natural environment, purified thus means having an increase in purity as a result of being separated from the other components of an original composition. The term “purified nucleic acid” describes a nucleic acid sequence that has been separated from other compounds including, but not limited to polypeptides, lipids, and carbohydrates which it is ordinarily associated with in its natural state.

The term “complementary” refers to two nucleic acid molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” or “substantially homologous” sequence is a nucleotide sequence that differs from a reference sequence only by one or more conservative substitutions, or by one or more non-conservative substitutions, deletions, or insertions located at positions of the sequence that do not destroy or substantially alter the activity of the polypeptide encoded by the nucleic acid molecule. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” or “substantially homologous” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

Nucleotide positions of polymorphisms described herein are provided with reference to the corresponding position on the Cannabis sativa (assembly cs10) representative genome, provided as RefSeq assembly accession: GCF_900626175.2 on NCBI, loaded on 14 Feb. 2019, referred to herein as “cs10 reference genome” or “cs10 genome”.

### Methods of Identifying a QTL or Haplotype Responsible for the Distinct Sesquiterpene Trait and Molecular Markers Therefor

In some embodiments, methods are provided for identifying a QTL or haplotype responsible for the distinct sesquiterpene trait of interest, such as the sesquiterpene absence or sesquiterpene presence trait, and for selecting plants with the distinct sesquiterpene trait of interest. In some embodiments, the methods may comprise the steps of:
a. Identifying a plant that displays the distinct sesquiterpene trait of interest within a breeding program.
b. Establishing a population by crossing the identified plant to itself (selfing) or a recipient parent plant.
c. Genotyping the resultant F1 or subsequent populations, for example, by sequencing methods.
d. Performing association studies, including phenotyping and linkage analysis, to discover QTLs and/or polymorphisms contained within the QTL.
e. Optionally, identifying cannabis paralogs of previously characterized genes that may be involved in conferring the distinct sesquiterpene trait of interest.
f. Developing molecular markers that detect one or more polymorphisms linked to QTLs, alleles within these QTLs, or existing or induced polymorphisms.
g. Validating the molecular markers by determining the linkage disequilibrium between the marker and the distinct sesquiterpene trait of interest.

### Trait Development and Introgression

In some embodiments, methods are provided for marker assisted breeding (MAB) or marker assisted selection (MAS) of plants having a distinct sesquiterpene QTL or displaying the distinct sesquiterpene trait of interest. The methods may comprise the steps of:
a. Identifying a plant that displays the distinct sesquiterpene trait of interest or which contains a distinct sesquiterpene QTL associated with the distinct sesquiterpene trait of interest as defined herein.
b. Establishing a population by crossing the identified plant to itself (selfing) or another recipient parent plant.
c. Genotyping and phenotyping the resultant F1 or subsequent populations, for example, by sequencing methods.
d. Performing association studies, inputting phenotype and genotype information to identify genomic regions enriched with polymorphisms associated with the distinct sesquiterpene trait, to discover QTLs and/or polymorphisms contained within the QTL.
e. Optionally, identifying cannabis paralogs of previously characterized genes that may be involved in conferring the distinct sesquiterpene trait of interest.
f. Developing molecular markers that detect one or more polymorphisms linked to QTLs, alleles within these QTLs, or existing or induced polymorphisms.
g. Using the molecular markers when introgressing the QTLs or polymorphisms into new or existing cannabis varieties to select plants containing the distinct sesquiterpene haplotype or the distinct sesquiterpene trait of interest.

### QTLs and Marker Assisted Breeding

In some embodiments, during the breeding process, selection of plants displaying the distinct sesquiterpene trait may be based on molecular markers designed to detect polymorphisms linked to genomic regions that control the distinct sesquiterpene trait of interest by either an identified or an unidentified mechanism. Previously identified genetic mechanisms may, for example, have a direct or pleiotropic effect on sesquiterpene concentrations in a plant. In some embodiments, QTLs containing such elements are identified using association studies. Knowledge of the mode-of-action is not required for the functional use of these genomic regions in a breeding program. Identification of regions controlling unidentified mechanisms may be useful in obtaining plants with the distinct sesquiterpene trait of interest, based on identification of polymorphisms that are either linked to, or found within QTLs that are associated with the distinct sesquiterpene trait of interest using association studies.

### Construction of Breeding Populations

Breeding populations are the offspring of sexual reproduction events between two or more parents. The parent plants (F0) are crossed to create an F1 population each containing a chromosomal complement of each parent. In a subsequent cross (F2), recombination has occurred and allows for mostly independent segregation of traits in the offspring, and importantly, the reconstitution of recessive phenotypes that existed in only one of the parental lines.

According to some embodiments, QTLs that lead to the phenotype of the distinct sesquiterpene trait of interest are identified within synthetic populations of plants capable of revealing dominant, recessive, or complex traits. In one embodiment of the invention, a genetically diverse population of cannabis varieties, that are used to produce the synthetic population are integrated into a breeding program by unnatural processes. In some embodiments, these processes result in changes in the genomes of the plants. The changes may include, but are not limited to, mutations and rearrangements in the genomic sequences, duplication of the entire genome (polyploidy), or activation of movement of transposable elements which may inactivate, activate, or attenuate the activity of genes or genomic elements. According to one embodiment of the invention, the methods employed to integrate the plants into a breeding program include some or all of the following:
a. Growing plants in rich media or soils under artificial lighting;
b. Cloning of plants, often through a multitude of sub-cloning cycles;
C. Introduction of plants into in vitro, sterile growth environments, and subsequent removal to standard growth conditions;
d. Exposure to mutagens such as EMS, colchicine, silver nitrate, ethidium bromide, dinitroanilines, high concentrations of mono or poly-chromatic light sources;
e. Growing plants under highly stressful conditions which include restricted space, drought, pathogen challenge, atypical temperatures, and nutrient stresses.

### Distinct Sesquiterpene Trait of Interest Association Studies and QTL Identification

In some embodiments, the synthetic populations created are either the offspring of the sexual reproduction or clones of plants in the breeding program such that genetic material of individuals in the synthetic populations is derived from one, or two, or more plants from the breeding program.

In one embodiment, plants identified within the synthetic population as having a distinct sesquiterpene trait of interest may be used to create a structured population for the identification of the genetic locus responsible for the trait. The structured population may be created by crossing one (selfing) or more plants and recovering the seeds from those plants.

Plants in the structured population may be fully genotyped using genome sequencing to identify genetic markers for use in the association study (AS) database. Association mapping is a powerful technique used to detect QTLs specifically based on the statistical correlation between the phenotype and the genotype. In a population generated by crossing, the amount of linkage disequilibrium (LD) is reduced between genetic marker and the QTL as a function of genetic distance in cannabis varieties with similar genome structures. Simple association mapping is performed by biparental crosses of two closely related lines where one line has a phenotype of interest, and the other does not. In some embodiments, advanced population structures may be used, including nested association mapping (NAM) populations or multi-parent advanced generation inter-cross (MAGIC) populations, however it will be appreciated that other population structures can also be effectively used. Biparental, NAM, or MAGIC structured populations can be generated and offspring, at F1 or later generations, may be maintained by clonal propagation for a desired length of time. In some embodiments, QTLs may be identified using the high-density genetic marker database created by genotyping the founder lines and structured population lines. This marker database may be coupled with an extensive phenotypic trait characterization dataset, including, for example, the distinct sesquiterpene phenotype of the plants. Using the association studies described herein, together with accurate phenotyping, this method is able to identify genomic regions, QTLs and even specific genes or polymorphisms responsible for the distinct sesquiterpene trait of interest that is directly introduced into recipient lines. Polygenic phenotypes may also be identified using the methods described herein.

In one embodiment, the structured population is grown to the time of harvest. To characterize the phenotypes of the lines, they are clonally reproduced so the phenotypic data can be collected in feasible replicates.

### Genomic Selection

In some embodiments, during the breeding process, selection of plants by genomic selection (GS) may be conducted. Genomic selection is a method in plant breeding where the genome wide genetic potential of an individual is determined to predict breeding values for those individuals. In some embodiments, the accuracy of genomic selection is affected by the data used in a GS model including size of the training population, relationships between individuals, marker density, use of pedigree information, and inclusion of known QTLs.

In some embodiments, a QTL or a SNP known to be associated with a trait that contributes to selection criteria can improve the accuracy of genomic selection models. In some embodiments, a genomic selection model that incorporates distinct sesquiterpene concentrations can be improved by the inclusion of the distinct sesquiterpene QTL in the GS model. In some embodiments, the SNPs described in Tables 2 to 5 may be useful in a genomic selection model, for example where genotypes with unknown phenotypes are evaluated using an approach like a random forest algorithm for prediction of the distinct sesquiterpene trait, and particularly in combination, to improve the predictive power of the model.

### Molecular Markers to Detect Polymorphisms

As used herein, the term “marker” or “genetic marker” refers to any sequence comprising a particular polymorphism or haplotype described herein that is capable of detection. For example, a marker may be a binding site for a primer or set of primers that is designed for use in a PCR-based method to amplify and thus detect a polymorphism or haplotype. Alternatively, the marker may introduce a restriction enzyme recognition site, or result in the removal of a restriction enzyme recognition site. Plants can be screened for a particular trait based on the detection of one or more markers confirming the presence of the polymorphism. Marker detection systems that may be used in accordance with the present invention include, but are not limited to polymerase chain reaction (PCR) followed by sequencing, Kompetitive allele specific PCR (KASP), restriction fragment length polymorphisms (RFLPs) analysis, amplified fragment length polymorphisms (AFLPs), cleaved amplified polymorphic sequences (CAPS), or any other markers known in the art.

In some embodiments “molecular markers” refers to any marker detection system, including primers designed based on the context sequences for the polymorphisms identified herein, provided in Table 6. Such molecular markers may include PCR primers, or targeted sequencing primers such as those described in the examples below, more specifically the primers defined in Table 7.

For example, PCR primers may be designed that consist of a reverse primer and two forward primers that are homologous to the part of the genome that contains a polymorphism but differ in the 3′ nucleotide such that the one primer will preferentially bind to sequences containing the polymorphism and the other will bind to sequences lacking it. The three primers are used in single PCR reactions where each reaction contains DNA from a cannabis plant as a template. Fluorophores linked to the forward primers provide, after thermocycling, a different relative fluorescent signal for homozygous and heterozygous alleles containing the polymorphism and for those lacking the polymorphism, respectively.

In some embodiments, allele-specific primers may each harbor a unique tail sequence that corresponds with a universal FRET (fluorescence resonant energy transfer) cassette. For example, the primer specific to the SNP may be labelled with a FAM and the other specific primer with a HEX dye. During the PCR thermal cycling performed with these primers, the allele-specific primer binds to the genomic DNA template and elongates, so attaching the tail sequence to the newly synthesized strand. The complement of the allele-specific tail sequence is then generated during subsequent rounds of PCR, enabling the FRET cassette to bind to the DNA. Alleles are discriminated through the competitive binding of the two allele-specific forward primers. At the end of the PCR reaction a fluorescent plate is read using standard tools which may include RT-PCR devices with the capacity to detect florescent signals and is evaluated with commercial software.

If the genotype at a given polymorphism site is homozygous, one of the two possible fluorescent signals will be generated. If the genotype is heterozygous, a mixed fluorescent signal will be generated. By way of example, genomic DNA extracted from cannabis leaf tissue at seedling stage can be used as a template for PCR amplifications with reaction mixtures containing the three primers. Final fluorescent signals can be detected by a thermocycler and analysed using standard software for this purpose, which discriminates between individuals that are heterozygotes or homozygotes for either allele.

In some embodiments, molecular markers to one, two, or more of the SNPs in the haplotype can be used to identify the presence of the QTL and by association, the distinct sesquiterpene trait of interest.

Further, the QTL may include a number of individual polymorphisms in linkage disequilibrium, which constitute a haplotype and which, with high frequency, can be inherited from a donor parent plant as a unit. Therefore, in some embodiments, molecular markers can be utilized which have been designed to identify numerous polymorphisms which are in linkage disequilibrium with other polymorphisms, any of which can be used to effectively predict the phenotype of the offspring for the distinct sesquiterpene trait of interest.

According to some embodiments, any polymorphism in linkage disequilibrium with the distinct sesquiterpene QTL can be used to determine the distinct sesquiterpene haplotype in a breeding population of plants, as long as the polymorphism is unique to the distinct sesquiterpene trait of interest in the donor parent plant when compared to the recipient parent plant.

In some embodiments the desired trait is the sesquiterpene absence trait, and the donor parent plant may be a plant that has been genetically modified or selected to include a sesquiterpene absence QTL defined by a polymorphism conferring the sesquiterpene absence trait, for example any, some, or all of the polymorphisms defined in Tables 2 to 5 associated with the trait.

Alternatively, the desired trait may be the sesquiterpene presence trait, and the donor parent plant may be a plant that has been genetically modified or selected to include a sesquiterpene presence QTL defined by a polymorphism associated with the sesquiterpene presence trait, for example any, some, or all of the polymorphisms defined in Tables 2 to 5 associated with the trait.

In some embodiments, donor parent plants, as described above, are used as one of two parents to create breeding populations (F1) through sexual reproduction. In this embodiment, donor parent plants may be identified by detecting polymorphisms using the molecular markers as described above.

Methods for reproduction that are known in the art may be used. The donor parent plant provides the distinct sesquiterpene trait to the breeding population. The trait is made to segregate through the population (F2) through at least one additional crossing event of the offspring of the initial cross. This additional crossing event can be either a selfing of one of the offspring or a cross between two individuals, provided that each plant used in the F1 cross contains at least one copy of a desired QTL allele or haplotype.

In some embodiments, the distinct sesquiterpene allele or distinct sesquiterpene haplotype in plants to be used in the F1 cross is determined using the described molecular markers. In some embodiments, the resulting F2 progeny, or subsequent progeny, is/are screened for any of the polymorphisms associated with the distinct sesquiterpene trait of interest, and in particular polymorphisms associated with the sesquiterpene absence trait, described herein.

The plants at any generation can be produced by asexual means like cutting and cloning, or any method that yields a genetically identical offspring.

Production of Cannabis Spp. Plants Having the Sesquiterpene Absence Trait

In some embodiments, a Cannabis spp. plant that produces the terpenes α-eudesmol, β-eudesmol, epi-γ-eudesmol, and guaiol may be converted into a plant that does not, according to the methods of the present invention by providing a breeding population where the donor parent plant contains a sesquiterpene absence QTL associated with the sesquiterpene absence trait, and the recipient parent plant either displays a sesquiterpene presence trait or contains the sesquiterpene presence QTL.

In some embodiments, the sesquiterpene presence trait may be removed from a recipient parent plant by crossing it with a donor parent plant having the sesquiterpene absence QTL. In some embodiments, the donor parent plant has a sesquiterpene absence phenotype and contains a contiguous genomic sequence characterized by one or more of the polymorphisms of any of Tables 2 to 5 associated with the distinct sesquiterpene allele or distinct sesquiterpene haplotype conferring the sesquiterpene absence trait.

In some embodiments, the donor parent plant is any Cannabis spp. variety that is cross fertile with the recipient parent plant.

In some embodiments, MAS or MAB may be used in a method of backcrossing plants carrying the sesquiterpene absence trait to a recipient parent plant. For example, an F1 plant from a breeding population can be crossed again to the recipient parent plant. In some embodiments, this method is repeated.

In some embodiments, the resulting plant population is then screened for the sesquiterpene absence trait using MAS with molecular markers to identify progeny plants that contain one or more polymorphism, such as any of those described in Tables 2 to 5, indicating the presence of an allele of the QTL associated with the sesquiterpene absence phenotype. In another embodiment, the population of cannabis plants may be screened by any analytical methods known in the art to identify plants with desired characteristics, specifically the sesquiterpene absence trait.

Production of Cannabis Spp. Plants Having the Sesquiterpene Presence Trait

In some embodiments, a Cannabis spp. plant that has the sesquiterpene absence trait may be converted into a plant having a sesquiterpene presence trait according to the methods of the present invention by providing a breeding population where the donor parent plant contains a sesquiterpene presence QTL and the recipient parent plant either displays the sesquiterpene absence trait or contains the sesquiterpene absence QTL.

In some embodiments the sesquiterpene absence trait may be removed from a recipient parent plant by crossing it with a donor parent plant having the sesquiterpene presence QTL. In some embodiments, the donor parent plant produces the terpenes α-eudesmol, β-eudesmol, epi-γ-eudesmol, and guaiol and contains a contiguous genomic sequence characterized by one or more of the polymorphisms of Tables 2 to 5 associated with the distinct sesquiterpene allele or distinct sesquiterpene haplotype conferring the sesquiterpene presence trait.

In some embodiments, the donor parent plant is any Cannabis spp. variety that is cross fertile with the recipient parent plant.

In some embodiments, MAS or MAB may be used in a method of backcrossing plants carrying the sesquiterpene presence trait to a recipient parent plant. For example, an F1 plant from a breeding population can be crossed again to the recipient parent plant. In some embodiments, this method is repeated.

In some embodiments, the resulting plant population is then screened for the sesquiterpene presence trait using MAS with molecular markers to identify progeny plants that contain one or more polymorphism, such as any of those described Tables 2 to 5, indicating the presence of an allele of a QTL associated with the sesquiterpene presence phenotype. In another embodiment, the population of cannabis plants may be screened by any analytical methods known in the art to identify plants with desired characteristics, specifically the sesquiterpene presence trait.

### Methods to Genetically Engineer Plants to Achieve the Distinct Sesquiterpene Trait of Interest Using Mutagenesis or Gene Editing Techniques

Identifying QTLs and individual polymorphisms that correlate with a trait when measured in an F1, F2, or similar breeding population indicates the presence of one or more causative polymorphisms in close proximity to the polymorphism detected by the molecular marker. In some embodiments, the polymorphisms associated with the absence or presence of the distinct sesquiterpene trait are introduced into a plant by other means so that the trait can be introduced into plants that would not otherwise contain associated causative polymorphisms or removed from plants that would otherwise contain associated causative polymorphisms. For example, the polymorphisms detailed in Tables 2 to 5 are molecular markers that can be used to indicate the presence of a possible causative polymorphism, including a SNP that causes an amino acid substitution at position 147 and/or position 303 of a protein with homology to a germacrene D synthase (SEQ ID NO:62) encoded by the LOC115695864 gene, such as a SNP resulting in the amino acid substitution 147S>P and/or 303G>D with reference to SEQ ID NO:62.

The entire QTL or parts thereof which confer the sesquiterpene trait of interest described herein, or the genes or nucleic acid molecules described herein, may be introduced into the genome of a cannabis plant to obtain plants with a distinct sesquiterpene trait of interest, through a process of genetic modification known in the art, for example, but not limited to, heterologous gene expression using an expression cassette including a sequence encoding the QTL or part thereof, the gene(s), or the nucleic acids comprising them. The expression cassettes may contain all or part of the QTL or gene(s), including the possible causative polymorphisms resulting in an amino acid substitution at position 147 and/or position 303 of a protein with homology to a germacrene D synthase encoded by the LOC115695864 gene (SEQ ID NO:62), such as a SNP resulting in the amino acid substitution 147S>P and/or 303G>D with reference to SEQ ID NO:62.

The distinct sesquiterpene trait of interest described herein may be removed from, or introduced into, the genome of a cannabis plant to obtain plants that exclude or include the causative polymorphisms and the potential to display a desired distinct sesquiterpene trait of interest through processes of genetic modification known in the art, for example, but not limited to, CRISPR-Cas9 targeted gene editing, TILLING, non-targeted chemical mutagenesis using e.g., EMS.

The present invention further provides methods for producing a modified Cannabis spp. plant using genome editing or modification techniques. For example, genome editing can be achieved using sequence-specific nucleases (SSNs) the use of which results in chromosomal changes, such as nucleotide deletions, insertions or substitutions at specific genetic loci, particularly those associated with the distinct sesquiterpene trait of interest described in Tables 2 to 5 or causative polymorphisms linked thereto. Non limiting examples of SSNs include zinc finger nucleases (ZFNs), TAL effector nucleases (TALENs), meganucleases, and clustered regularly interspaced short palindromic repeats (CRISPR)/CRISPR-associated protein (Cas) system. In some embodiments, non-limiting examples of Cas proteins suitable for use in the methods of the present invention include Csnl, Cpf1 Cas9, Cas 12, Cas 13, Cas 14, CasX and combinations thereof. In one embodiment, a modified Cannabis spp. plant having a distinct sesquiterpene trait of interest is generated using CRISPR/Cas9 technology, which is based on the Cas9 DNA nuclease guided to a specific DNA target by a single guide RNA (sgRNA). For example, the genome modification may be introduced using guide RNA, e.g., single guide RNA (sgRNA) designed and targeted to introduce a polymorphism associated with the distinct sesquiterpene trait of interest described in Tables 2 to 5, or a causative polymorphism linked thereto.

DNA introduction into the plant cells can be performed using Agrobacterium infiltration, virus-based plasmid delivery of the genome editing molecules and mechanical insertion of DNA (PEG mediated DNA transformation, biolistics, etc.). In some embodiments, the Cas9 protein may be directly inserted together with a gRNA (ribonucleoprotein-RNP's) in order to bypass the need for in vivo transcription and translation of the Cas9+gRNA plasmid in planta to achieve gene editing. In one embodiment, a genome edited plant may be developed and used as a rootstock, so that the Cas protein and gRNA can be transported via the vasculature system to the top of the plant and create the genome editing event in the scion.

According to one embodiment of the present invention, the method of genetically modifying a plant may be achieved by combining the Cas nuclease (e.g., Cas9, Cpf 1) with a predefined guide RNA molecule (gRNA). The gRNA is complementary to a specific DNA sequence targeted for editing in the plant genome and which guides the Cas nuclease to a specific nucleotide sequence. The gRNA may be designed based on the context sequence for the polymorphisms provided in Table 6. The predefined gene-specific gRNAs may be cloned into the same plasmid as the Cas gene and this plasmid is inserted into plant cells as described above.

In some embodiments, once the gRNA molecule and Cas9 nuclease reach the specific predetermined DNA sequence, the Cas9 nuclease cleaves both DNA strands to create double stranded breaks leaving blunt ends. This cleavage site is then repaired by the cellular non homologous end joining DNA repair mechanism resulting in insertions or deletions which introduce a mutation at the cleavage site.

In one embodiment, a deletion form of the mutation may consist of at least 1 base pair deletion. As a result of this base pair deletion the gene coding sequence for a gene responsible for the distinct sesquiterpene trait of interest, such as the LOC115695864 gene encoding a protein with homology to a germacrene D synthase, the LOC115695865 gene encoding a terpene synthase, and/or the LOC115695866 gene encoding a terpene synthase, with reference to Table 8, is disrupted and the translation of the encoded protein is compromised either by a premature stop codon or disruption of a functional or structural property of the protein.

In another embodiment, the distinct sesquiterpene trait of interest in Cannabis spp. plants may be introduced by generating gRNA with homology to a specific site of predetermined genes in the Cannabis genome or the QTL defined herein. In one embodiment the gene may be the LOC115695864 gene encoding a protein with homology to a germacrene D synthase, the LOC115695865 gene encoding a terpene synthase, and/or the LOC115695866 gene encoding a terpene synthase, with reference to Table 8. This gRNA may be sub-cloned into a plasmid containing the Cas9 gene, and the plasmid inserted into the Cannabis plant cells. In this way site specific mutations in the QTL are generated, particularly causative polymorphisms linked to a SNP associated with the distinct sesquiterpene trait of interest described in Tables 2 to 5, and in particular a SNP that causes an amino acid substitution at position 147 and/or position 303 of a protein with homology to a germacrene D synthase (SEQ ID NO:62) encoded by the LOC115695864 gene, such as a SNP resulting in the amino acid substitution 147S>P and/or 303G>D with reference to SEQ ID NO:62, thus effectively introducing the distinct sesquiterpene trait of interest into the genome edited plant.

In some embodiments, a modified Cannabis spp. plant exhibiting a sesquiterpene absence trait may be obtained using the targeted genome modification methods described above, wherein the plant comprises a targeted genome modification to introduce one or more polymorphisms associated with the sesquiterpene absence trait, particularly a causative polymorphism linked to a SNP defined in Tables 2 to 5, wherein the modification effects the sesquiterpene absence trait.

In some embodiments, the genetic modification may be introduced using gene silencing, a process by which the expression of a specific gene product is lessened or attenuated. Gene silencing can take place by a variety of pathways, including by RNA interference (RNAi), an RNA dependent gene silencing process. In one embodiment, RNAi may be achieved by the introduction of small RNA molecules, including small interfering RNA (siRNA), microRNA (miRNA) or short hairpin RNA (shRNA), which act in concert with host proteins (e.g., the RNA induced silencing complex, RISC) to degrade messenger RNA (mRNA) in a sequence-dependent fashion. In particular, RNAi may be used to silence the LOC115695864 gene encoding a protein with homology to a germacrene D synthase, the LOC115695865 gene encoding a terpene synthase, and/or the LOC115695866 gene encoding a terpene synthase, with reference to Table 8. Such RNAi molecules may be designed based on the sequence of these genes. These molecules can vary in length (generally 18-30 base pairs) and may contain varying degrees of complementarity to their target mRNA in the antisense strand. Some, but not all, RNAi molecules have unpaired overhanging bases on the 5′ or 3′ end of the sense strand and/or the antisense strand. As used herein, the term “RNAi molecule” includes duplexes of two separate strands, as well as single strands that can form hairpin structures comprising a duplex region. The RNAi molecules may be encoded by DNA contained in an expression cassette and incorporated into a vector. The vector may be introduced into a plant cell using Agrobacterium infiltration, virus-based plasmid delivery of the vector containing the expression cassette and/or mechanical insertion of the vector (PEG mediated DNA transformation, biolistics, etc.).

Plants may be screened with molecular markers as described herein to identify transgenic individuals with the distinct sesquiterpene trait of interest or having a distinct sesquiterpene QTL or polymorphism(s), following the genetic modification.

In some embodiments, Cannabis spp. plants having one or more of the polymorphisms of Tables 2 to 5 associated with the distinct sesquiterpene QTL or a causative polymorphism linked thereto are provided. The polymorphisms, including the possible causative polymorphisms resulting in an amino acid substitution at position 147 and/or position 303 of a protein with homology to a germacrene D synthase (SEQ ID NO: 62) encoded by the LOC115695864 gene, such as a SNP resulting in the amino acid substitution 147S>P and/or 303G>D with reference to SEQ ID NO:62, or the genes, such as the LOC115695864 gene encoding a protein with homology to a germacrene D synthase, the LOC115695865 gene encoding a terpene synthase, and/or the LOC115695866 gene encoding a terpene synthase, with reference to Table 8, may be introduced, for example, by genetic engineering. In some embodiments the one or more polymorphisms associated with the distinct sesquiterpene trait of interest, or a causative polymorphism linked thereto are introduced into the plants by breeding, such as by MAS or MAB, for example as described herein.

The distinct sesquiterpene QTL herein, or genes identified herein responsible for effecting the distinct sesquiterpene trait, may be under the control of, or operably linked to, a promoter, for example an inducible promoter. Such QTL or genes may be operably linked to the inducible promoter so as to induce or suppress the distinct sesquiterpene trait or phenotype in the plant or plant cell.

Accordingly, in a further embodiment, Cannabis spp. plants comprising a distinct sesquiterpene QTL described herein, including a sesquiterpene absence QTL or a sesquiterpene presence QTL, or one or more polymorphisms associated therewith, are provided. In some cases, such plants are provided for with the proviso that the plant is not exclusively obtained by means of an essentially biological process.

The following examples are offered by way of illustration and not by way of limitation.

### Example 1

### Genome-Wide Association Studies (GWAS) of Sesquiterpene Levels in Cannabis

The inventors undertook a survey of sesquiterpene levels in a diverse population of cannabis flowers, including hemp-type and resin-type plants. Plants were originally assembled and grown in a field trial in 2020 in Niederwil, Switzerland. The inventors noticed a large diversity of aromas in this diverse population. Many of the plants with distinct aromas were used to generate F2 populations. During outdoor field trials in 2021, 24 of these F2 populations were grown to maturity and characterized for specific sesquiterpene levels in harvested dried flower.

At the time of harvest, in mid-October 2021, flowers were harvested from the primary flowering stems, dried, freeze-dried, and analysed for their constituent terpene content.

The total terpene profile and amount was determined on an Agilent 8890 GC system equipped with a flame ionization detector (FID) and head space sampler. One or two flower pieces were homogenised with a hand grinder, and a 20-30 mg aliquot weighed in a 20 ml GC glass vial. Alternatively, for exhaustive extraction of all terpenes, 500 mg of ground cannabis flower was extracted in 5 ml of ethanol (99.6%, Ph.Eur. grade) under 10 min of sonication and 30 μl of extract were added to the 20 ml GC glass vial. In the headspace sampler, samples were heated to 130° C. for 10 min before the terpene-containing gas phase was injected for analysis. Separation of terpenes was achieved on an Agilent DB HeavyWAX (30 m×250 μm×0.5 μm), using hydrogen as carrier gas.

Instrument control, data acquisition, and integration were achieved with OpenLAB CDS (Agilent Technologies) software, applying an identification and quantification method based on a 5-level external standards calibration curve. To confirm the analyte identity in plant material, retention time was compared with the signal acquired on certified reference materials (CRMs).

The calibration curve used for quantification was obtained by analysing serial dilutions of a CRM terpene mix (SPEX Certiprep, PART #: CAN-TERP-KIT-H, Can-Terp Kit (High Level), 1000 μg/mL (1000 ppm) in methanol containing 42 different terpenes usually present in cannabis flowers. Additional single terpenes were acquired as authentic references to identify them, using the average response factor for monoterpenes or sesquiterpenes from the 42 terpene calibration for their quantification. The terpenes were: alpha-pinene, camphene, beta-pinene, sabinene, 3-carene, beta-myrcene, alpha-phellandrene, alpha-terpinene, D-limonene, eucalyptol, beta-phellandrene, beta-ocimene (2 isomers), gamma-terpinene, p-cymene, terpinolene, fenchone, limonene-1.2-epoxide, sabinene hydrate, camphor, linalool, linalyl acetate, alpha-cedrene, isopulegol, fenchol, bornyl acetate, isobornyl acetate, citral (4 isomers), beta-caryophyllene, terpinene-4-ol, menthol, pulegone, all-trans-beta-farnesene, isoborneol, alpha-humulene, alpha-terpineol, borneol, valencene, alpha-farnesene, neryl acetate, beta-bisabolene, geranyl acetate, citronellol, beta-maaliene, alpha-bisabolene, selina-3.7 (11)-diene, nerol, geraniol, caryophyllene oxide, cis-nerolidol, trans-nerolidol, guaiol, epi-gamma-eudesmol, cedrol, alpha-bisabolol, alpha-eudesmol, beta-eudesmol, phytol (2 isomers). The content of terpenes is calculated in % of the dry mass of cannabis flower (% w/w).

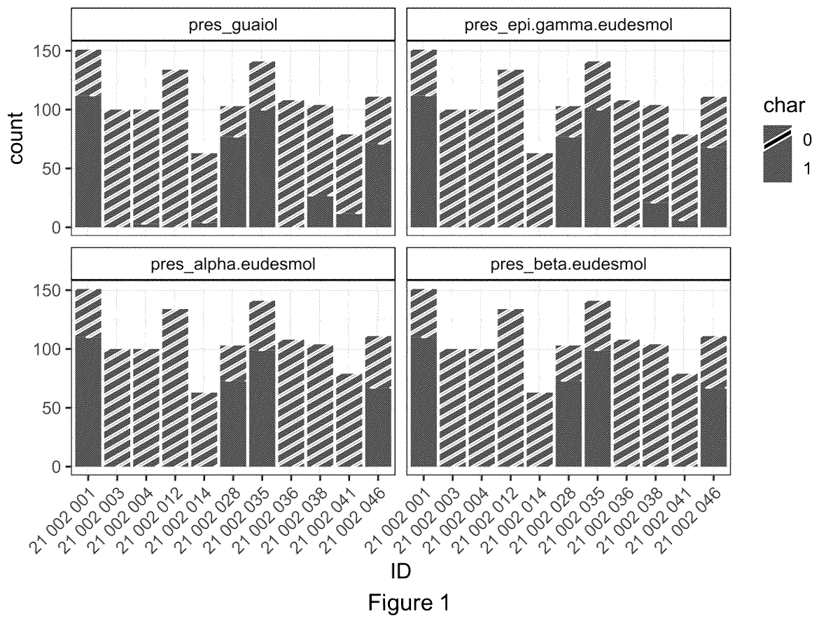

The inventors sought to understand the genetic basis of sesquiterpene presence or absence in the 24 segregating populations. In other studies that have looked to understand the genetics of terpenes, abundance rather than the presence or absence of a terpene is used as an input for GWA studies. This can be limiting as it can result in the identification of genes that may regulate the amount of terpenes rather than their presence or absence. Instead, the inventors sought to identify genomic regions associated with the absolute presence or absence of the sesquiterpenes measured. To do this the inventors conducted a survey to qualify the presence or absence of terpenes across the 24 populations examined (Table 1 and FIG. 1). The inventors identified a unreported phenomenon whereby guaiol, together with α-eudesmol, β-eudesmol, and epi-γ-eudesmol are not present in plants comprising some of the populations under study e.g., in population 21002003, 21002004, 21002012, 21002014, and 21002036. Conversely, in the 21002001, 21002028, 21002035, and 21002046 F2 populations the presence or absence of these sesquiterpenes seemed to be segregating (Table 1 and FIG. 1).

TABLE 1
A representative overview of the F2 populations used in determining presence
or absence of guaiol, α-eudesmol, β-eudesmol, and epi-γ-eudesmol
(given as % of population), including the population identifier (ID), as
well as the sample size of the population given by the number of plants (Number).

| | | | | alpha- | | beta- | | epi-gamma- | |
|---|---|---|---|---|---|---|---|---|---|
| | | guaiol | | eudesmol | | eudesmol | | eudesmol | |
| | | % | % | % | % | % | % | % | % |
| ID | Number | present | absent | present | absent | present | absent | present | absent |
| 21 002 001 | 151 | 74 | 26 | 73 | 27 | 73 | 27 | 74 | 26 |
| 21 002 003 | 101 | 0 | 100 | 0 | 100 | 0 | 100 | 0 | 100 |
| 21 002 004 | 110 | 8 | 92 | 0 | 100 | 0 | 100 | 0 | 100 |
| 21 002 011 | 158 | 66 | 34 | 66 | 34 | 66 | 34 | 66 | 34 |
| 21 002 012 | 134 | 0 | 100 | 0 | 100 | 0 | 100 | 0 | 100 |
| 21 002 014 | 71 | 13 | 87 | 0 | 100 | 0 | 100 | 0 | 100 |
| 21 002 028 | 107 | 76 | 24 | 72 | 28 | 72 | 28 | 76 | 24 |
| 21 002 035 | 141 | 71 | 29 | 70 | 30 | 70 | 30 | 71 | 29 |
| 21 002 036 | 108 | 0 | 100 | 0 | 100 | 0 | 100 | 0 | 100 |
| 21 002 038 | 105 | 27 | 73 | 0 | 100 | 0 | 100 | 21 | 79 |
| 21 002 041 | 79 | 15 | 85 | 0 | 100 | 0 | 100 | 8 | 92 |
| 21 002 046 | 112 | 64 | 36 | 61 | 39 | 61 | 39 | 62 | 38 |

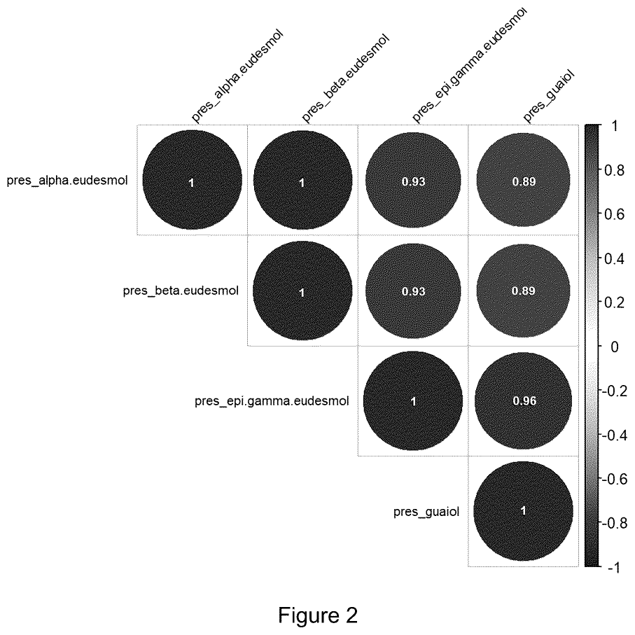

The inventors conducted a correlation study to examine the correlation of the presence of guaiol, α-eudesmol, β-eudesmol, and epi-γ-eudesmol. The inventors found that these four sesquiterpenes are strongly positively correlated (FIG. 2). It is possible that α-eudesmol, β-eudesmol, and epi-γ-eudesmol may be synthesized by a common terpene synthase, and although structurally distinct, guaiol may share a common biosynthetic intermediate (FIG. 3). To better understand the genetic regulation of these sesquiterpenes the inventors carried out a GWA study using their presence and absence as input.

DNA from 1377 plants, a subset of the F2 plants, was sequenced. DNA was extracted from about 70 mg of leaf discs from all the plants evaluated using an adapted kit with “sbeadex” magnetic beads by LGC Genomics, which was automated on a KingFisher Flex with 96 Deep-Well Head by Thermo Fisher Scientific.

The extracted DNA served as a template for the subsequent library preparation for sequencing. The library pools were prepared according to the manufacturer's instructions (AgriSeq™ HTS Library Kit—96 sample procedure from Thermo Fisher Scientific). Targeted sequencing of a custom SNP marker panel based on the Cannabis sativa CS10 reference genome was carried out on the Ion Torrent system by Thermo Fisher Scientific. The primers for the SNPs identified are provided in Table 7 below. The library pool was loaded onto Ion 550 chips with Ion Chef and sequenced with Ion GeneStudio S5 Plus according to the manufacturer's instructions (Ion 550™ Kit from Thermo Fisher Scientific).

Using a subset of the 24 combined F2 populations with a total of 1337 individuals after filtering, genome-wide association studies (GWAS) were performed to detect significant associations between genotypic information derived from targeted resequencing of the custom SNP marker panel described above with sesquiterpene presence or absence of guaiol, α-eudesmol, β-eudesmol, and epi-γ-eudesmol, where presence=1, and absence=0 was used as input for the GWA.

For better modelling the inventors reasoned that a high rate of missing values may be impacting the estimation of population structure and kinship among individuals. To solve this, an additional step was incorporated. The GWA with all F2 populations combined was performed again but instead using a SNP matrix that underwent a round of imputation for reducing the number of missing values. In order to reduce missing data in the genotype file, an imputation was performed using the HapMap_imputation software (GitHub-mwylerCH/HapMap_Imputation). Briefly, the genotype file was converted to a hapmap format (comma separated, http://augustogarcia.me/statgen-esalq/Hapmap-and-VCF-formats-and-its-integration-with-onemap/#hapmap).

In a first step, HapMap_Imputation counts the occurrence of each nucleotide at every single genotyped position. The most common nucleotide is defined as major allele, the second is defined as minor allele. Missing genotyping information is excluded. In case major and minor alleles occur at the same number, the nucleotide of the reference cs10 genome is chosen as the major allele. Subsequently, HapMap_Imputation sorts markers by position and parses the hapmap into the required fastPHASE (Scheet and Stephens, 2006) input format. Briefly, HapMap_Imputation splits the haplotypes into two separate rows, converts major and minor alleles into 0 and 1, respectively, and produces temporary files for each chromosome.

During the third step, HapMap_Imputation downloads the latest fastPHASE version and runs the imputation using 8 cores in parallel. fastPHASE is run with ten random starts of the imputation algorithm. After imputation, HapMap_Imputation reverses the 0 and 1 coding into the major and minor nucleotide, respectively. Subsequently, the two haplotypes are combined, and the separate chromosomes are merged into a single file.

The genotypic matrix was filtered for SNPs having more than 30% missing values within the population and a minor allele frequency lower than 5%. This resulted in 5077 SNP markers after filtering. The GWAS was performed using GAPIT version 3 (Wang and Zhang, 2021) with four statistical models: General Linear Model (GLM), Mixed Linear Model (MLM), FarmCPU and Blink. A quantile-quantile plot (QQ plot) was used to evaluate the statistical models. The MLM model performed the best by the inventors' evaluation for the GWA of presence vs absence of guaiol, α-eudesmol, β-eudesmol, and epi-γ-eudesmol and were used for the analysis. SNPs surpassing a LOD (−log₁₀(p-value)) value of 5 were considered to have a significant association with trait variation.

Unexpectedly, using presence and absence of the four sesquiterpenes, the inventors were able to identify SNPs with an LOD value greater than 5 in the MLM model in the imputed GWA, with reference to the Cannabis Sativa CS10 genome (Table 2, Table 3, Table 4, Table 5) showing a significant association with guaiol, «-eudesmol, β-eudesmol, and epi-γ-eudesmol. Surprisingly, the inventors discovered that guaiol, α-eudesmol, β-eudesmol, and epi-γ-eudesmol share a common QTL on NC_044377.1 in approximately the region of position 75059527-78081084, with reference to the CS10 genome, that was found to be associated with the presence or absence of these terpenes. The SNP “common_4528” was found by the inventors to have the highest LOD score in all except guaiol. “Common_4528” was found as a SNP significantly associated with the presence/absence of guaiol as well, though it was not the SNP with the highest LOD score. In all cases, “common_4528” when in the allele variant GG predicts for the absence of guaiol, α-eudesmol, β-eudesmol, and epi-γ-eudesmol. Alternatively, the “common_4528” AA allele variant predicts for the presence of guaiol, α-eudesmol, β-eudesmol, and epi-γ-eudesmol. “Common_4528”, therefore, is an excellent marker to select for or against the presence of guaiol, α-eudesmol, β-eudesmol, and epi-γ-eudesmol. In the guaiol experiment, the SNP with the highest LOD score was “common_4518”, which was also a significantly associated SNP in the other GWA. As such, “common_4518” is also an excellent marker to select for or against the presence of guaiol, α-eudesmol, β-eudesmol, and epi-γ-eudesmol. The “common_4518” allelic variant AA is strongly predictive for the presence of these four sesquiterpenes as is the heterozygous state AG, while the GG state of this allele strongly predicts the absence of these four sesquiterpenes (Table 2, Table 3, Table 4, Table 5).

The reference or context sequence for each of the SNPs identified is provided in Table 6 with reference to the CS10 genome. In Table 7, PCR primers designed to amplify each of the regions containing these SNPs, with reference to the CS10 genome, are provided for the allelic variant to be determined.

Tables 2 to 5: SNPs associated with guaiol (Table 2), α-eudesmol (Table 3), β-eudesmol (Table 4) and epi-γ-eudesmol (Table 5) in the F2 populations. Mean percent presence of guaiol/α-eudesmol/β-eudesmol/epi-γ-eudesmol is predicted by the occurrence of the indicative allele (marked with *). The positions of the SNPs are provided with reference to the CS10 reference genome. All of the polymorphisms occurred on Chromosome NC_044377.1 with reference to the CS10 reference genome. The LOD score for the MLM model is provided as LOD. Mean_1, Mean_2 and Mean_3 denote the average phenotypic value associated with the allelic variants provided as Allele_1, Allele_2 and Allele_3, respectively, based on mean percent presence of guaiol/α-eudesmol/β-eudesmol/epi-γ-eudesmol. Count_1, Count_2, and Count_3 denote the number of plants that contributed to the average phenotypic value of Mean_1, Mean_2, and Mean_3, respectively. The possible alleles for each of the SNPs is also provided (Alleles).

TABLE 2
SNPs associated with guaiol in the F2 populations

| SNP | Position | Alleles_1 | Alleles_2 | Alleles_3 | Mean_1 | Mean_2 |
|---|---|---|---|---|---|---|
| common_4518 | 75977377 | AA* | AG | GG | 0.96031746 | 0.89230769 |
| common_4517 | 75907527 | AA* | AC | CC | 0.96610169 | 0.87545788 |
| common_4519 | 76201790 | AA | AG | GG* | 0.08675799 | 0.64075067 |
| common_4528 | 76959457 | AA* | AG | GG | 0.7137931 | 0.37556561 |
| common_4525 | 76757669 | AA | AC | CC* | 0.1097561 | 0.4524237 |
| GBScompat_common_881 | 78081084 | AA | AG | GG* | 0.08774373 | 0.592 |
| GBScompat_common_880 | 77726400 | AA* | AG | GG | 0.88268156 | 0.77419355 |
| GBScompat_common_878 | 76764685 | AA | AG | GG* | 0.2855407 | 0.46590909 |
| GBScompat_rare_169 | 77047478 | AA* | AG | GG | 0.93814433 | 0.875 |
| GBScompat_common_877 | 76400764 | AA* | AG | GG | 0.8021978 | 0.58947368 |
| common_4490 | 73291051 | AA* | AG | GG | 0.88198758 | 0.81818182 |
| common_4510 | 75324947 | CC | CG | GG* | 0.25655022 | 0.53370787 |

| SNP | Mean_3 | Count_1 | Count_2 | Count_3 | LOD | Alleles |
|---|---|---|---|---|---|---|
| common_4518 | 0.08353808 | 126 | 260 | 814 | 19.7454656 | G/A/C/T |
| common_4517 | 0.08405439 | 118 | 273 | 809 | 19.5982546 | A/C |
| common_4519 | 0.73529412 | 657 | 373 | 170 | 18.5656842 | A/G |
| common_4528 | 0.1025641 | 290 | 442 | 468 | 18.1162797 | G/A |
| common_4525 | 0.53218884 | 410 | 557 | 233 | 14.5161934 | C/A |
| GBScompat_common_881 | 0.90517241 | 718 | 250 | 232 | 13.6093062 | A/G |
| GBScompat_common_880 | 0.24141414 | 179 | 31 | 990 | 11.5402049 | G/A/C/T |
| GBScompat_common_878 | 0.55752212 | 823 | 264 | 113 | 11.1143209 | A/G |
| GBScompat_rare_169 | 0.23923924 | 97 | 104 | 999 | 10.4043029 | G/A/C/T |
| GBScompat_common_877 | 0.25680087 | 91 | 190 | 919 | 8.51320402 | A/G |
| common_4490 | 0.25663717 | 161 | 22 | 1017 | 5.57004705 | G/A |
| common_4510 | 0.85849057 | 916 | 178 | 106 | 5.18439569 | G/C |

TABLE 3
SNPs associated with α-eudesmol on the F2 populations

| SNP | Position | Alleles_1 | Alleles_2 | Alleles_3 | Mean_1 | Mean_2 |
|---|---|---|---|---|---|---|
| common_4528 | 76959457 | AA* | AG | GG | 0.69655172 | 0.33710407 |
| common 4525 | 76757669 | AA | AC | CC* | 0.00485437 | 0.41218638 |
| common_4519 | 76201790 | AA* | AG | GG | 0.00603318 | 0.6155914 |
| common_4518 | 75977377 | AA* | AG | GG | 0.96031746 | 0.87258687 |
| common_4517 | 75907527 | AA* | AC | CC | 0.96610169 | 0.85294118 |
| GBScompat_common_880 | 77726400 | AA* | AG | GG | 0.88268156 | 0.73333333 |
| GBScompat_rare_169 | 77047478 | AA* | AG | GG | 0.94845361 | 0.86407767 |
| GBScompat_common_881 | 78081084 | AA | AG | GG* | 0.01793103 | 0.54216867 |
| GBScompat_common_878 | 76764685 | AA | AG | GG* | 0.21237864 | 0.43609023 |
| common_4527 | 76935328 | AA* | AG | GG | 0.4025974 | 0.25335893 |

| SNP | Mean_3 | Count_1 | Count 2 | Count_3 | LOD | Alleles |
|---|---|---|---|---|---|---|
| common_4528 | 0.00632911 | 290 | 442 | 474 | 20.1193811 | G/A |
| common 4525 | 0.51694915 | 412 | 558 | 236 | 19.7821645 | C/A |
| common_4519 | 0.70760234 | 663 | 372 | 171 | 16.2863243 | A/G |
| common_4518 | 0.00852619 | 126 | 259 | 821 | 14.8385729 | G/A/C/T |
| common_4517 | 0.00980392 | 118 | 272 | 816 | 14.6916309 | A/C |
| GBScompat_common_880 | 0.17452357 | 179 | 30 | 997 | 10.4812076 | G/A/C/T |
| GBScompat_rare_169 | 0.17196819 | 97 | 103 | 1006 | 10.055438 | G/A/C/T |
| GBScompat_common_881 | 0.88793103 | 725 | 249 | 232 | 9.71923233 | A/G |
| GBScompat_common_878 | 0.54310345 | 824 | 266 | 116 | 7.15717491 | A/G |
| common_4527 | 0.30131827 | 154 | 521 | 531 | 5.80028239 | A/G |

TABLE 4
SNPs associated with β-eudesmol on the F2 populations

| SNP | Position | Alleles_1 | Alleles_2 | Alleles_3 | Mean_1 | Mean_2 |
|---|---|---|---|---|---|---|
| common_4528 | 76959457 | AA* | AG | GG | 0.69655172 | 0.33786848 |
| common_4525 | 76757669 | AA | AC | CC* | 0.00485437 | 0.41292639 |
| common_4519 | 76201790 | AA | AG | GG* | 0.0060423 | 0.61725067 |
| common_4518 | 75977377 | AA* | AG | GG | 0.96031746 | 0.87596899 |
| common_4517 | 75907527 | AA* | AC | CC | 0.96610169 | 0.85608856 |
| GBScompat_common_880 | 77726400 | AA* | AG | GG | 0.88268156 | 0.73333333 |
| GBScompat_rare_169 | 77047478 | AA* | AG | GG | 0.94845361 | 0.86407767 |
| GBScompat_common_881 | 78081084 | AA | AG | GG* | 0.01798064 | 0.54216867 |
| GBScompat_common_878 | 76764685 | AA | AG | GG* | 0.21237864 | 0.43773585 |
| common_4527 | 76935328 | AA* | AG | GG | 0.4025974 | 0.25433526 |
| GBScompat_common_882 | 78973740 | AA* | AG | GG | 0.56302521 | 0.26315789 |
| common_4510 | 75324947 | CC | CG | GG* | 0.19456522 | 0.49438202 |

| SNP | Mean_3 | Count_1 | Count_2 | Count_3 | LOD | Alleles |
|---|---|---|---|---|---|---|
| common_4528 | 0.00634249 | 290 | 441 | 473 | 19.8029844 | G/A |
| common_4525 | 0.51914894 | 412 | 557 | 235 | 19.6052261 | C/A |
| common_4519 | 0.70760234 | 662 | 371 | 171 | 16.01372 | A/G |
| common_4518 | 0.00853659 | 126 | 258 | 820 | 14.6676058 | G/A/C/T |
| common_4517 | 0.00981595 | 118 | 271 | 815 | 14.5769844 | A/C |
| GBScompat_common_880 | 0.17487437 | 179 | 30 | 995 | 10.4477598 | G/A/C/T |
| GBScompat_rare_169 | 0.17231076 | 97 | 103 | 1004 | 10.1133285 | G/A/C/T |
| GBScompat_common_881 | 0.88793103 | 723 | 249 | 232 | 9.60519401 | A/G |
| GBScompat_common_878 | 0.54782609 | 824 | 265 | 115 | 7.06727016 | A/G |
| common_4527 | 0.30131827 | 154 | 519 | 531 | 5.74125258 | A/G |
| GBScompat_common_882 | 0.02702703 | 357 | 551 | 296 | 5.40705138 | A/G/T/C |
| common_4510 | 0.82075472 | 920 | 178 | 106 | 5.05433308 | G/C |

TABLE 5
SNPs associated with epi-γ-eudesmol on the F2 populations

| SNP | Position | Alleles_1 | Alleles_2 | Alleles_3 | Mean_1 | Mean_2 |
|---|---|---|---|---|---|---|
| common_4528 | 76959457 | AA* | AG | GG | 0.70934256 | 0.35535308 |
| common_4519 | 76201790 | AA | AG | GG* | 0.05038168 | 0.63243243 |
| common_4525 | 76757669 | AA* | AC | CC | 0.06203474 | 0.43345324 |
| common_4517 | 75907527 | AA* | AC | CC | 0.96610169 | 0.875 |
| common_4518 | 75977377 | AA* | AG | GG | 0.96031746 | 0.89189189 |
| GBScompat_rare_169 | 77047478 | AA* | AG | GG | 0.93814433 | 0.875 |
| GBScompat_common_878 | 76764685 | AA | AG | GG* | 0.2496925 | 0.46037736 |
| GBScompat_common_880 | 77726400 | AA* | AG | GG | 0.87709497 | 0.77419355 |
| GBScompat_common_881 | 78081084 | AA | AG | GG* | 0.05874126 | 0.56680162 |
| GBScompat_common_877 | 76400764 | AA* | AG | GG | 0.79347826 | 0.58421053 |
| common_4527 | 76935328 | AA* | AG | GG | 0.41447368 | 0.27539063 |
| common_4490 | 73291051 | AA* | AG | GG | 0.88198758 | 0.81818182 |

| SNP | Mean_3 | Count_1 | Count_2 | Count_3 | LOD | Alleles |
|---|---|---|---|---|---|---|
| common_4528 | 0.05818966 | 289 | 439 | 464 | 22.3881921 | G/A |
| common_4519 | 0.7245509 | 655 | 370 | 167 | 19.5784081 | A/G |
| common_4525 | 0.52360515 | 403 | 556 | 233 | 19.0708629 | C/A |
| common_4517 | 0.04488778 | 118 | 272 | 802 | 18.4519813 | A/C |
| common_4518 | 0.04460967 | 126 | 259 | 807 | 18.2459434 | G/A/C/T |
| GBScompat_rare_169 | 0.20787084 | 197 | 104 | 991 | 10.7534476 | G/A/C/T |
| GBScompat_common_878 | 0.55263158 | 813 | 265 | 114 | 10.6009 | A/G |
| GBScompat_common_880 | 0.2107943 | 179 | 31 | 982 | 10.5069353 | G/A/C/T |
| GBScompat_common_881 | 0.89565217 | 715 | 247 | 230 | 9.79196277 | A/G |
| GBScompat_common_877 | 0.22417582 | 92 | 190 | 910 | 7.52964438 | A/G |
| common_4527 | 0.34848485 | 152 | 512 | 528 | 6.13382703 | A/G |
| common_4490 | 0.2259663 | 161 | 22 | 1009 | 5.36791685 | G/A |

TABLE 6
Detailed information of each of the SNPs listed in Tables 2-5 is provided. The “Ref”
reference allele based on the CS10 genome, and the identified “Alt” alternative allele
based on the SNP marker panel are given for each SNP. The “context sequence” is provided
with the SNP given in brackets. All the sequences and alleles are provided with reference
to the plus strand.

| SNP | Ref | Alt | Context sequence |
|---|---|---|---|
| GBScompat_ | C | T | GCTATATTGGCTATATTCAAACGTTTTGATTTTGAATAGGTACTGGTCCCATGT |
| rare_169 | | | GGTGAATTGCCCCAGTTGCAGTGCAGCATACAAAGCTCTGAATGTGGTTAAA |
| | | | GTTGTGCTCAGTATCACCTCTCTTGTTTTGATTGGTCTTCTGGGTGTGACCAA |
| | | | TAAGCTAGCCACTGCATCAGTTGCTTTGAGAGCCACAATGGT[C/T]TCACTCT |
| | | | CAATACTTTGCTTTGCAGCCTCAAAATGGCTGAATCACTTTGTTTACAAGAACT |
| | | | TTCACTTTCATGACTATATTCATGCTTTTCATTAATTAACACTACAAATATATAT |
| | | | TGTACATATATAATTCAATGTCATCAGAAGAAGGAAGAGCTTCTGTTATTACAC |
| | | | ACAAACACACAATTAAGTATAAAAAGACAGC |
| | | | (SEQ ID NO: 1) |
| GBScompat_ | T | C | TTTAAATATTTCAAGATGTGGGCTTCACATCTTGAATACAATACAATGGCCAAA |
| common_882 | | | GATGTTTAGTAGCTAGCAAACTGTTATAGGGACCAGAATGTATCAGGTCATAT |
| | | | CCAAGTTTAAGGCTCTCAAAAGTGTCTTCAAAGAGATGATCAATACAGAAGGA |
| | | | TTTCTGATATCCAAGCTGCTAAATACATGCTAAGGAGATTC[T/C]GGTGAAATG |
| | | | TCACAATAAACTACATCTTGATCCCATTTCTGGGAAAATTCTCAGGGAGATCA |
| | | | ACTATAATGTACTAACACTTATCCCCAAAACTAAATGCCCTAATACAGTAAGTG |
| | | | AATTTAAGCTTATAGCATGCTGCAATTTGATCTACAAAATAGCTACCAAGGTG |
| | | | ATTTGCTCAAGGCTCATATACATACTGCCTAA |
| | | | (SEQ ID NO: 2) |
| GBScompat_ | G | A | GAATGTCTCTTTTCATATTGTGCTATTGGTGTTGTCCATAAGAGAATTTCCCTC |
| common_881 | | | TCTTTGAAGTTTTCAGTGATTTATATGCAGAACCTTTGATCAGGCCATTTGTGG |
| | | | GTTAAGAGGCATGTTGTGATTTTTCAAGTGTGTCATTGTTGAATACATAAAAG |
| | | | GGTCTCCAAAGCAGCAGCTAGCTACTTCAACACACTTTGT[G/A]ACGATTATGT |
| | | | TGTTGGTCTGTATTTATGGCACTACTACTGATGAAACAAATTCGAGGTCAACT |
| | | | CACTTGGTGTAGCAGTTCATTGAGATTTCGTGTGGCTTCAGCTACTTCTTGGA |
| | | | ATGCCTTGGTATCAACTCAAATTCGAGAATTATATCTCAGTAGAACTATCAAAC |
| | | | ATTTTTCTACTAGTGTTGAGAGTGAAAAGCT |
| | | | (SEQ ID NO: 3) |
| GBScompat_ | C | T | TCGGAAAGCAAAAGCAGCTAGGTCCTCTTACGCTGCTGCATCGATGGTAGTT |
| common_880 | | | GATGCTGTTGAAAATGATGGTAAATCCACTTGTGTTCTTGGTTCCAGGCCAAC |
| | | | TTCGAGCCATGAAGAATCTAGCAAAGGAGGTTCGGAATATGATAGACCTTTTA |
| | | | AAATCTCTTTAGTGTCTTATGAGGAGTATTTAAGAGATCGGAA[C/T]TTTGTGA |
| | | | AACTCGATACTGAAGCAAAGGAAGTGCTAAATACCATGTTAGCTAAGCTGCA |
| | | | GACTCAAGTTGAATTGTTAACATCTGAACGAGGTATTATCCAACCAACTGAGG |
| | | | AAATTTTGGTGAAATTAGTCAAGGCATTTCTAGAAGCAGGAAAGGTTAAAGAT |
| | | | TTAGCCGAGTTTCTCATCAAGGTGGAGAGAGAAGAT |
| | | | (SEQ ID NO: 4) |
| GBScompat_ | G | A | AAACAGTTCGTAAATGTATGTATTTATAGGCAAGGTCGGGTGGCTCGGTGGA |
| common_878 | | | CCTGAATTGTTAAGAACACGTCGGATCAAGTTATTATTGAATGTCTCAGCATTT |
| | | | TCAAGATTGGCATCCGGTTCTTTGGCACCACCGAGCCATGGCCAACCAGOTT |
| | | | CGGTAACAACAACAGGAATCCCCGAAAAATTTGCAGCCTCCAT[G/A]GCATAA |
| | | | TAGGTAGCATCCACCATTGCATCAAACATGCTACTATAGTGGAAAAGAGTGTT |
| | | | GGGATCAACAATCTGCTTAACCGAGGGAAGTGGTCGAAAAATAGCATAATCA |
| | | | ATCGGAAAAATGCCATCCCCTTCCGTGTATCCATAATATGGATACGCATTTAA |
| | | | CATGTAATAGGAATTTGTGTTTTTCAAAAACTGAAGG |
| | | | (SEQ ID NO: 5) |
| GBScompat_ | G | A | AGAGGAAGCTAGTGACGAAGATGCACACTTGACATAAACCGAAATCAACGAA |
| common_877 | | | TTCAAAACCGAAGTAACAAAACACAATCCGAACTTCACAACCGCGCAATGCAT |
| | | | TTGTTGACACTGCCTTTCATCATCAACTACAAGTCCAAAAGCACTAAGAACAC |
| | | | TAGATAAAGTAAAGTCATCAACCCCAACACTAACCCTCCTCAT[G/A]TCATGAA |
| | | | ATGTTTGAATAGCAGCAAAGCCATCATTGTTATGAGAACAACAAGTGATCATG |
| | | | GCATTGTAGAAAACAGTGTCTCTCATGGTCAATGGAGTAGCAAAGAACACTTC |
| | | | CCTTGCCAACTTAAGATTCCCAGCTACAGAGTAAGCTGCAATCATAGTCGTTC |
| | | | TAGAAATGATATCTGGTTTAGGAATTTGGTCGAAC |
| | | | (SEQ ID NO: 6) |
| common_4528 | A | G | AAGTAAATAATATTTACATAAGTGAATTAGAATGAAGTAATAAGCAATAAAGTG |
| | | | CCACAAACTCCAACAAATCCAACTACAAATCCCACTCCAATTCCCATATAAAG |
| | | | CCATGACATGTTAAGCCACCCTTCATCATATTCTTCAGCATCACTTGGATCAT |
| | | | GAATATCTTTAGGGCTATTTGGTGTCTCATCTCCAAGACAT[A/G]AATTGTTTA |
| | | | GTGGAAGTCCGCACAATCCATCATTATCAATGTATATCGAAGCATTAAAACTT |
| | | | TGCAATTGAGTACCGATAGGAATTCTTCCAGACAATTTGTTACTTGACAAATTC |
| | | | AAAAAAGATAGAGAAGATATACTTGCCAAGCTTGTTGGAATGACACTAGAAAG |
| | | | CTTGTTATGGGATAAATCTAGAGAATCCAACT |
| | | | (SEQ ID NO: 7) |
| common_4527 | A | G | AGAGGACTAAAATACTTGCATCAAGATTGTAGTGCGGCGATTTTACATTTTGA |
| | | | CATAAAACCTCATAACATTCTTCTAGATTTTGATTTTTCTCCTAAAATTTCTGAC |
| | | | TTCGGCCTTGCCAAGTTGTGGCAAAGGGATGGGAGTGGTGTATCACTGTTGA |
| | | | AGGGTAGAGGCACAATTGGATATACAGCACCGGAGATGCAT[A/G]ACAGAAAT |
| | | | TTTGGTGAAGTATCTTATAAATCTAATGTTTATAGTTATGGAATGATGGTTTTA |
| | | | GAAATGGTGGGCGGAAGAAAAAATATTGATACTAGTGTTTCTCGAACAAGTG |
| | | | CAATATATTATCCACATTGGGCACATAAGCATATCAATGATGATGATGATGGT |
| | | | GAGCTCTTGAAAAATATTTGTGAAGAAATAATGG |
| | | | (SEQ ID NO: 8) |
| common_4525 | T | C | TTTATATTACCTACATGAAATCAGACAACAACAGCATATCGGGGGCTTCACTC |
| | | | ATGGAGATGGAGACCTACTACCATTTGGTTCATCTGGGTAGCGATTTGGACTT |
| | | | CGGCTAAGGCTTCTCTTTGGGCTTCGGCTTCTGCTTCGACTATGGCTTGGAC |
| | | | TTCTAGCTCCACTTGGACCCCTAGCTGGGCTTCGACTTGGGCT[T/C]CTTGAT |
| | | | CCATTATATGGTGGAGAACGAGAGTACGACCTGTCTCGGCTGTACCTTCTCT |
| | | | CCTGTGGAGACACAGATCTGGCCCGAAGAGGGAGTACGAAAATTAATGTTTA |
| | | | AACATTGAAATATATTAAATGCATGTTTTCTCCTATTGCTAAAGATCCCACATT |
| | | | TTTAATGCTGACTAGAGAAGTTGAAAAGATATACTTG |
| | | | (SEQ ID NO: 9) |
| common_4519 | A | G | CGATCACTTCGTAGATGCATCCTCCCACAAGGTAGCACAATTGTAGAAAGTG |
| | | | CTAAATCATGCTTTATTCCATTTGTTCTTTTTGTCTTCTCTTTTTGOTTAATCGA |
| | | | ACGATGTTGTGAACTTGTAGGGTTGTCAAATTCGAAGGGAGAGCGCACATGG |
| | | | AGAGAGCGTTTGTTGCAACAATGTGAGGGCCCTGTTTGATGA[A/G]CTCCCAA |
| | | | CTCCACACCTAATTGTGGAGATCACACCATTCCCTGAAGGGCCTCTCACTGA |
| | | | AAAAGATTACACCAAAGCTGAGAAATTGGAGAGGGTACTTAGAACTGGCCCG |
| | | | AACGTTTGATTCTTCTCTCGAGTTAAATCATCGCTGTCTCTCGTTAGAACTACA |
| | | | GOTTAATTGTATGTATGTTTTGAGCCTTGTACATAT |
| | | | (SEQ ID NO: 10) |
| common_4518 | C | T | TTTTATTTCTCTCACTTTTTTTGCATACTCTTTTCTTTCCATTCTTCTCGATCGT |
| | | | GCTGAAATACCTAATAAGACAGTGACACAGCATGGCATGACACAATTAATGG |
| | | | GAGCGGTTGCCTTTGCACAACAACTCCTCCTCTTCCACCTCCACTCTGCTGAT |
| | | | CATATGGGACCAGAGGGACAATATCACTTGCTACTCCAGCT[C/T]GTGATTCT |
| | | | TGTCTCTCTGGTCACATCTCTAATGGGAATAGGGCTACCGAAGAGTTTCTTAG |
| | | | TGAGTTTTGTTAGGTCTCTTAGCATTTTGTTTCAAGGGGTTTGGCTTATGGTG |
| | | | ATGGGGTTTATGTTATGGACACCATCCTTGATTTCCAAAGGGTGTTTCATGCA |
| | | | CTATGAGGAAGGTCATCATGTGGTGAGATGCTCA |
| | | | (SEQ ID NO: 11) |
| common_4517 | C | A | CACAGCATCTCCATTAGAAGGATTAAACTTATGAGTACTAGCATGAGCATACC |
| | | | CACTTGCAAACCTAAAAAGACCACTTCCACCAATCACAGGCATTTCTCTAACC |
| | | | TTATCAAACACTTGGTTTCTACCAAGAATGGTGAGAGTGCTCCCATTGTATTT |
| | | | CCCTTGAGTTATATGAAAATTCATAGCCATAATTAGGGCAAT[C/A]TCTTCTTGT |
| | | | GAAGCTAATCCATAAAACCCTTGAGCTTTTCCTAGCAACTTTGAGCTTACTTCT |
| | | | GGCCCTTCTGTCAATGGATTGTCGATCATGCTAACCGCCCCGAACCCACTTT |
| | | | TCGATGCATTGGCCGGTGGTTGGATTATTGCCATCGCGCTAGGGTTTTTGCC |
| | | | GCTGTATATGTCGTGCCAATAGAACCGAAAGTGG |
| | | | (SEQ ID NO: 12) |
| common_4510 | C | G | GCTAACAAGTTAAACACCAAGATACAAATTCTACCACATCTTTCTTCATCCTGC |
| | | | ACAACTAGTAGAAGGGTCTTAAATCTTGATACATCTTGGTAGTGCCAAGATGC |
| | | | AGTGAGTATGGAGTGGTATGAGCTTCTTTGAACACAAACTCTGGTTTTATACA |
| | | | ATAGCATCCCATTACTTGATACCGTAAAATATTTAGTTTGA[C/G]CAGCTAAAA |
| | | | CCCCTTCCCAGATCTTTACCAGTTCTGGATCAGTTAACTGAGCAGCTTTAATT |
| | | | CTGTCTAAAAAACCAGATTGCAATGTCAAATTATGAAGTTTGCCTATGACAAA |
| | | | CTCAATGCCTACTCTGACCGTATCATCTGCTAGCTGAGGGGAAATCTTAATCA |
| | | | TACTAGTTACCCGTCCAGGATCCTTTTTGCTCA |
| | | | (SEQ ID NO: 13) |
| common_4490 | A | G | ACTTATCTTCCTTCCTAAGTCCCTGCCTGCCGCTAACTTTTACCAAAGCCTTA |
| | | | AAAAAAAATTGTTCAAAGTCCTGAAATTCCAAAACTACCCTTACAAAAAAAAAT |
| | | | AATATTCTTTTTTCCTCAAAAACTACCCTTATTATTATTTTTTTCCTCAAAAACTT |
| | | | GAAATTACGCGGGCCAACCCGGATCCTGACCCGGGTCC[A/G]GGTCCGGGT |
| | | | CCGGATCCGACAACTTCCTTCTTCTTCCTCATTTCCAAAACTTTCCGATGACT |
| | | | ATTCGAATGGACCTCGCTGGAAAAAGTCGGACTACAAGCGGGTCGGTACTCG |
| | | | GGGAAAAGACGACCCGACTTGAACCGAACCCCACAAGCGTTACAAAGGGTTT |
| | | | TGGGACCGAGCGGACCGGTTCGCCACTGTGGTGTC |
| | | | (SEQ ID NO: 14) |

TABLE 7
Targeted sequencing primers (5′ to 3′) for the SNPs identified in Tables 2-5, as described
in Example 1.

| SNP | Forward Primer 1 | Reverse Primer 1 | Forward Primer 2 | Reverse Primer 2 |
|---|---|---|---|---|
| GBScompat_ | CCAGTTGCAGTG | AAGCTCTTCCTT | CCAGTTGCAGTG | AGCTCTTCCTTC |
| rare_169 | CAGCATAC | CTTCTGATGACA | CAGCATAC | TTCTGATGACA |
| | (SEQ ID NO: 15) | (SEQ ID NO: 16) | (SEQ ID NO: 15) | (SEQ ID NO: 17) |
| GBScompat_ | AGGCTCTCAAAA | GCCTTGAGCAAA | AAGGCTCTCAAA | GCCTTGAGCAAA |
| common_882 | GTGTCTTCA | TCACCTTGG | AGTGTCTTCA | TCACCTTGG |
| | (SEQ ID NO: 18) | (SEQ ID NO: 19) | (SEQ ID NO: 20) | (SEQ ID NO: 19) |
| GBScompat_ | TTGTGCTATTGG | AAGTAGCTGAAG | TGTGCTATTGGT | AGTAGCTGAAGC |
| common_881 | TGTTGTCCA | CCACACGA | GTTGTCCA | CACACGAA |
| | (SEQ ID NO: 21) | (SEQ ID NO: 22) | (SEQ ID NO: 23) | (SEQ ID NO: 24) |
| GBScompat_ | GCCAACTTCGAG | CACCTTGATGAG | GCCAACTTCGAG | ACCTTGATGAGA |
| common_880 | CCATGAAG | AAACTCGGC | CCATGAAG | AACTCGGCT |
| | (SEQ ID NO: 25) | (SEQ ID NO: 26) | (SEQ ID NO: 25) | (SEQ ID NO: 27) |
| GBScompat_ | GCTCGGTGGAC | CACGGAAGGGG | GCTCGGTGGAC | ACGGAAGGGGA |
| common_878 | CTGAATTGT | ATGGCATTT | CTGAATTGT | TGGCATTTT |
| | (SEQ ID NO: 28) | (SEQ ID NO: 29) | (SEQ ID NO: 28) | (SEQ ID NO: 30) |
| GBScompat_ | GAACTTCACAAC | AGCTGGGAATCT | GAACTTCACAAC | AGTTGGCAAGGG |
| common_877 | CGCGCAAT | TAAGTTGGCA | CGCGCAAT | AAGTGTTCT |
| | (SEQ ID NO: 31) | (SEQ ID NO: 32) | (SEQ ID NO: 31) | (SEQ ID NO: 33) |
| common_4528 | ACAAATCCCACT | GTCATTCCAACA | ACAAATCCCACT | TTCCAACAAGCT |
| | CCAATTCCCA | AGCTTGGCA | CCAATTCCCA | TGGCAAGT |
| | (SEQ ID NO: 34) | (SEQ ID NO: 35) | (SEQ ID NO: 34) | (SEQ ID NO: 36) |
| common_4527 | TCAAGATTGTAG | TTCTTCCGCCCA | CATCAAGATTGT | TTCTTCCGCCCA |
| | TGCGGCGA | CCATTTCT | AGTGCGGCG | CCATTTCT |
| | (SEQ ID NO: 37) | (SEQ ID NO: 38) | (SEQ ID NO: 39) | (SEQ ID NO: 38) |
| common_4525 | CAAGTGCTTGCT | GCGCTACAGGCT | AGTGCTTGCTGA | GCGCTACAGGCT |
| | GAGGATGG | CTCAGAAT | GGATGGAT | CTCAGAAT |
| | (SEQ ID NO: 40) | (SEQ ID NO: 41) | (SEQ ID NO: 42) | (SEQ ID NO: 41) |
| common_4519 | GATGCATCCTCC | CGGGCCAGTTCT | TCCTCCCACAAG | ACGTTCGGGCCA |
| | CACAAGGT | AAGTACCC | GTAGCACA | GTTCTAAG |
| | (SEQ ID NO: 43) | (SEQ ID NO: 44) | (SEQ ID NO: 45) | (SEQ ID NO: 46) |
| common_4518 | TAATGGGAGCGG | GCATCTCACCAC | GACACAGCATGG | AGTGCATGAAAC |
| | TTGCCTTT | ATGATGACC | CATGACAC | ACCCTTTGG |
| | (SEQ ID NO: 47) | (SEQ ID NO: 48) | (SEQ ID NO: 49) | (SEQ ID NO: 50) |
| common_4517 | GCATGAGCATAC | CGGCCAATGCAT | ACTTCCACCAAT | CAAAAACCCTAG |
| | CCACTTGC | CGAAAAGT | CACAGGCA | CGCGATGG |
| | (SEQ ID NO: 51) | (SEQ ID NO: 52) | (SEQ ID NO: 53) | (SEQ ID NO: 54) |
| common_4510 | AGTGCCAAGATG | AGGATCCTGGAC | AGTGCCAAGATG | AGGATCCTGGAC |
| | CAGTGAGT | GGGTAACT | CAGTGAGT | GGGTAACTA |
| | (SEQ ID NO: 55) | (SEQ ID NO: 56) | (SEQ ID NO: 55) | (SEQ ID NO: 57) |
| common_4490 | GCCTGCCGCTAA | CTTTTCCCCGAG | CTTCCTAAGTCC | CCCGCTTGTAGT |
| | CTTTTACC | TACCGACC | CTGCCTGC | CCGACTTT |
| | (SEQ ID NO: 58) | (SEQ ID NO: 59) | (SEQ ID NO: 60) | (SEQ ID NO: 61) |

### Example 2

### Gene Identification

Many of the terpene synthase genes in cannabis have been described based on amino acid sequence comparison to known terpene synthase proteins. Several of these terpene synthase proteins have been tested in in vitro assays. To the inventors' knowledge the mechanism and gene or genes involved in regulating the coregulation of the presence or absence of guaiol, α-eudesmol, β-eudesmol, and epi-γ-eudesmol is unknown. In Example 1 markers are provided to identify plants with or without guaiol, α-eudesmol, β-eudesmol, and epi-γ-eudesmol. The inventors searched the region of the QTL identified in CS10 but were unable to resolve which gene, and in particular, what causative SNP is responsible for the phenomenon under investigation. The inventors considered terpene synthase genes good candidates, however, there were over 13 potential genes that encoded for potential terpene synthase proteins. The inventors sought to use the findings of the association studies to identify candidate genes at the QTL identified through comparing a collection of cannabis genomic sequences to identify putative causative SNPs that appeared in a similar pattern as the polymorphisms identified in Tables 2 to 5.

The inventors determined that SNP differences between cannabis genomes could inform which genes play a role in the trait of interest. Short reads from sequenced lines were dereplicated with NGSReads Treatment (version 1.3, Gaia et al. (2019)) and pre-processed with fastp (version 0.23.2, S. Chen et al. (2018)). Reads were aligned to the CS10 reference genome with Bowtie2 (version 2.3.5.1, with options--rg and --rg-id to add read-group identifiers, Langmead and Salzberg (2012)). Only unique alignments with a mapping quality of at least 10 were kept. SNPs were called with freebayes and filtered for a minimal quality of 20 (version v1.3.2-40-gcce27fc, parameters -p 2--min-coverage 20-g 30000--min-alternate-count 4--min-alternate-fraction 0.1--min-mapping-quality 10--max-complex-gap-1, Garrison and Marth (2012)). SNPs were finally filtered for a coverage between 5 and 10,000 within each line and annotated with snpEff (version 4_3t, Cingolani et al. (2012)).

For each line, a pseudogenome was constructed by incorporating its variants into the CS10 reference genome with vcf-consensus (Danecek et al. (2011)). CS10 annotation was lifted over to align genes from a reference genome to a target genome with liftoff (version 1.6.3, Shumate and Salzberg (2021)). Protein and cDNA sequences were extracted with custom scripts. Proteins and cDNA sequences for a given protein/transcript from all lines were aligned with muscle (v3.8.31, Edgar (2004)).

Proteins on chromosome NC_044377.1 located between 75059527-78081084 bp were extracted. Multiple alignments from protein sequences were converted to tables including the variant positions and impact of SNP variants on protein composition. The variant position was tested for correlation with the significant SNP “common_4528” from the GWA marker panel and assigned an FDR score based on the correlation. Only proteins with significant FDR score were kept. The remaining 23 SNPs and 23 associated proteins with homologs in Arabidopsis were finally used as candidates (Table 8). These were further filtered for functional candidates. A gene, LOC115695864, that encodes a protein annotated as an alpha-humulene synthase was identified as a top candidate. Within this gene, the variants associated with differences in the distinct sesquiterpene trait allowed the identification of causal polymorphisms, detected by being significantly associated with “common_4528.” The first at position 76675473, results in an amino acid substitution of 147S>P, where the underlying SNP changes from C>T (CCA>TCA). The change in amino acid from S to P or P to S is significant enough to alter the protein's function, either by changes to the structure or the function of the protein. Inspection of the protein product of LOC115695864 in comparison to other protein variants in the inventors collection surprisingly identified a second causal polymorphism GGT>GAT that results in an amino acid substitution of G>D at amino acid position 303 of the protein product from LOC115695864. The 303G>D amino acid change occurs in a highly conserved residue amongst sesquiterpene type synthases where, when the amino acid is G, the enzyme loses activity-reported for similar enzymes. Because of the proximity to LOC115695864 and because they are reported to encode terpene synthases the inventors consider the genes LOC115695865 and LOC115695866 causal gene candidates as well.

TABLE 8
Gene candidate table based on association with “common_4528”. SNP position is the SNP tested based on reference genome
lines, all positions are provided with reference to the CS10 reference genome position on the NC_044377.1 chromosome, SNPfdr is
the association of the tested SNP with “common_4528”, SNP effect gives the impact of the SNP on the protein sequence.
The gene ID and protein ID are provided with reference to the CS10 reference genome and protein description is given based on Arabidopsis.

| SNP | | | | | |
|---|---|---|---|---|---|
| position | SNPfdr | SNPeffect | Gene ID | Protein ID | Protein Description |
| 76959458 | 2.36E−14 | MODERATE; missense_variant; L/S | LOC115695538 | XP_030478456.1 | LRR receptor-like serine/threonine-protein |
| | | | | | kinase GSO2 [0.4] |
| 76932130 | 6.67E−07 | MODERATE; missense_variant; E/Q | LOC115695537 | XP_030478455.1 | Protein kinase domain-containing protein [0.37] |
| 76675473 | 6.67E−07 | MODERATE; missense_variant; S/P | LOC115695864 | XP_030478815.1 | (−)-germacrene D synthase [0.7] |
| 76763694 | 1.29E−06 | MODERATE; missense_variant; Y/F | LOC115725595 | XP_030511035.1 | Glucan endo-1,3-beta-glucosidase 4 [0.55] |
| 76763694 | 1.29E−06 | MODERATE; missense_variant; Y/F | LOC115725595 | XP_030511036.1 | Glucan endo-1,3-beta-glucosidase 4 [0.55] |
| 76763694 | 1.29E−06 | MODERATE; missense_variant; Y/F | LOC115725595 | XP_030511037.1 | Glucan endo-1,3-beta-glucosidase 4 [0.55] |
| 76532752 | 2.42E−06 | MODERATE; missense_variant; V/I | LOC115695884 | XP_030478843.1 | none |
| 76843264 | 2.52E−05 | HIGH; frameshift_variant; | LOC115695532 | XP_030478449.1 | Receptor-like protein EIX2 [0.52]; |
| | | CIKN/CIKKX | | | LRR domain containing protein |
| | | | | | (Fragment) [0.39] |
| 76783997 | 2.52E−05 | MODERATE; missense_variant; K/E | LOC115724873 | XP_030510093.1 | LRR receptor-like serine/threonine-protein |
| | | | | | kinase GSO1 [0.44] |
| 76783997 | 2.52E−05 | MODERATE; missense_variant; K/E | LOC115724873 | XP_030510094.1 | LRR receptor-like serine/threonine-protein |
| | | | | | kinase GSO1 [0.44] |
| 76783997 | 2.52E−05 | MODERATE; missense_variant; K/E | LOC115724873 | XP_030510095.1 | NA |
| 76791485 | 3.09E−05 | MODERATE; missense_variant; V/A | LOC115724876 | XP_030510101.1 | LRR receptor-like serine/threonine-protein |
| | | | | | kinase GSO2 [0.43] |
| 76771569 | 7.27E−05 | HIGH; stop_lost; */S | LOC115695692 | XP_030478617.1 | Ent-kaurene oxidase, chloroplastic [0.9] |
| 76991393 | 9.51E−05 | MODERATE; missense_variant; R/Q | LOC115724452 | XP_030509600.1 | Zinc finger-domain containing protein [0.26] |
| 76809022 | 0.0001226 | MODERATE; missense_variant; R/K | LOC115724683 | XP_030509881.1 | Receptor like protein 42 [0.42]; |
| 76985259 | 0.0001226 | MODERATE; missense_variant; E/D | LOC115725006 | XP_030510263.1 | NA |
| 77059863 | 0.0001226 | MODERATE; inframe_deletion; | LOC115725568 | XP_030510997.1 | Protochlorophyllide-dependent translocon |
| | | SLLL/SLL | | | component 52, chloroplastic [0.86] |
| 76905057 | 0.0001499 | MODERATE; missense_variant; N/D | LOC115695534 | XP_030478451.1 | NA |
| 76969139 | 0.0004237 | MODERATE; missense_variant; R/C | LOC115724752 | XP_030509949.1 | LRR domain containing protein |
| | | | | | (Fragment) [0.46] |
| 78192986 | 0.0004418 | MODERATE; missense_variant; L/S | LOC115695582 | XP_030478499.1 | NA |
| 76613806 | 0.0008254 | MODERATE; missense_variant; R/K | LOC115695870 | XP_030478822.1 | PORR domain-containing protein [0.61]; |
| | | | | | hydrolase family protein [0.37]; Vacuolar- |
| | | | | | processing enzyme [0.19] |
| 77175789 | 0.0008943 | MODERATE; missense_variant; L/F | LOC115694718 | XP_030477666.1 | Leucine-rich repeat receptor-like protein |
| | | | | | FASCIATED EAR2 (Fragment) [0.49] |

1. A method for characterizing a Cannabis spp. plant with respect to a distinct sesquiterpene trait, the method comprising the steps of:
(i) genotyping at least one plant with respect to a distinct sesquiterpene QTL by detecting one or more polymorphisms associated with the distinct sesquiterpene trait as defined in any of Tables 2 to 5; and
(ii) characterizing the one or more plants with respect to the distinct sesquiterpene QTL as having a sesquiterpene absence QTL or a sesquiterpene presence QTL based on the genotype at the polymorphism.

2. The method of claim 1, wherein the polymorphism is selected from the group consisting of “common_4518”, “common_4528”, and combinations thereof, as defined in any of Tables 2 to 5.

3. The method of claim 1, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

4. The method of claim 3, wherein the molecular markers are for detecting polymorphisms at regular intervals within the distinct sesquiterpene QTL such that recombination can be excluded, or wherein the molecular markers are for detecting polymorphisms at regular intervals within the distinct sesquiterpene QTL such that recombination can be quantified to estimate linkage disequilibrium between a particular polymorphism and the distinct sesquiterpene phenotype, optionally wherein the molecular markers are designed based on a context sequence for the polymorphism in Table 6 or are selected from the primer pairs as defined in Table 7.

5. (canceled)

6. (canceled)

7. The method of claim 1, wherein the distinct sesquiterpene QTL is a quantitative trait locus having a sequence that corresponds to nucleotides 75059527-78081084 of NC_044377.1 of the CS10 genome and defined by one or more polymorphisms associated with the distinct sesquiterpene trait as defined in any of Tables 2 to 5, or a genetic marker linked to the QTL.

8. A method of producing a Cannabis spp. plant having a distinct sesquiterpene trait of interest, the method comprising the steps of:
(i) providing a donor parent plant having in its genome a distinct sesquiterpene QTL characterized by one or more polymorphisms associated with the distinct sesquiterpene trait of interest as defined in any of Tables 2 to 5;
(ii) crossing the donor parent plant having the distinct sesquiterpene QTL with at least one recipient parent plant to obtain a progeny population of cannabis plants;
(iii) screening the progeny population of cannabis plants for the presence of the distinct sesquiterpene QTL; and
(iv) selecting one or more progeny plants having the distinct sesquiterpene QTL, wherein the mature plant displays the distinct sesquiterpene trait of interest.

9. The method of claim 8, further comprising:
(v) crossing the one or more progeny plants with the donor recipient plant; or
(vi) selfing the one or more progeny plants.

10. The method of claim 8, wherein the screening comprises genotyping at least one plant from the progeny population with respect to the distinct sesquiterpene QTL by detecting one or more polymorphisms associated with the distinct sesquiterpene trait of interest as defined in any of Tables 2 to 5, and optionally wherein the method further comprises a step of genotyping the donor parent plant with respect to the distinct sesquiterpene QTL by detecting one or more polymorphisms associated with the distinct sesquiterpene trait of interest as defined in any of Tables 2 to 5, prior to step (i).

11. (canceled)

12. The method of claim 10, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

13. The method of claim 12, wherein the molecular markers are for detecting polymorphisms at regular intervals within the distinct sesquiterpene QTL such that recombination can be excluded or such that recombination can be quantified to estimate linkage disequilibrium between a particular polymorphism and the distinct sesquiterpene trait of interest, optionally wherein the molecular markers are designed based on a context sequence for the polymorphism in Table 6 or are selected from the primer pairs as defined in Table 7.

14. (canceled)

15. The method of claim 8, wherein the distinct sesquiterpene QTL is a sesquiterpene absence QTL or a sesquiterpene presence QTL.

16. The method of claim 8, wherein the polymorphism is selected from the group consisting of “common_4518”, “common_4528”, and combinations thereof, as defined in any of Tables 2 to 5.

17. The method of claim 8, wherein the distinct sesquiterpene QTL is a quantitative trait locus having a sequence that corresponds to nucleotides 75059527-78081084 of NC_044377.1 of the CS10 genome and defined by one or more polymorphisms associated with the distinct sesquiterpene trait as defined in any of Tables 2 to 5, or a genetic marker linked to the QTL.

18. A method of producing a Cannabis spp. plant that has a distinct sesquiterpene trait of interest, the method comprising introducing a distinct sesquiterpene QTL characterized by one or more polymorphisms associated with the distinct sesquiterpene trait of interest as defined in any of Tables 2 to 5 into a Cannabis spp. plant, wherein said QTL is associated with the distinct sesquiterpene trait of interest in the plant.

19. The method of claim 18, wherein introducing the distinct sesquiterpene QTL comprises crossing a donor parent plant having the distinct sesquiterpene QTL characterized by one or more polymorphisms associated with the distinct sesquiterpene trait of interest with a recipient parent plant.

20. The method of claim 18, wherein introducing the distinct sesquiterpene QTL characterized by one or more polymorphisms associated with distinct sesquiterpene trait of interest comprises genetically modifying the Cannabis spp. plant.

21. The method of claim 18, wherein the distinct sesquiterpene QTL is a quantitative trait locus having a sequence that corresponds to nucleotides 75059527-78081084 of NC_044377.1 of the CS10 genome and is defined by one or more polymorphisms associated with the distinct sesquiterpene trait as defined in any of Tables 2 to 5, or a genetic marker linked to the QTL.

22. (canceled)

23. (canceled)

24. (canceled)

25. A Cannabis spp. plant comprising a distinct sesquiterpene QTL characterized by one or more polymorphisms associated with a distinct sesquiterpene trait of interest as defined in any of Tables 2 to 5, wherein said distinct sesquiterpene QTL is associated with the distinct sesquiterpene trait of interest in the plant.

26. (canceled)

27. (canceled)

28. A plant extract obtained from a Cannabis spp. plant of claim 25.

29. An isolated nucleic acid comprising a gene that controls a distinct sesquiterpene trait in a Cannabis spp. plant, wherein the gene is selected from the group consisting of LOC115695864 encoding a protein with homology to a germacrene D synthase, LOC115695865 encoding a terpene synthase, and LOC115695866 encoding a terpene synthase, with reference to Table 8.

30. The isolated nucleic acid of claim 28, wherein the gene is LOC115695864 encoding a protein with homology to a germacrene D synthase, having a single nucleotide polymorphism resulting in an amino acid substitution at position 147 and/or position 303 of SEQ ID NO:62, optionally wherein the single nucleotide polymorphism results in an amino acid substitution of 147S>P and/or 303G>D of SEQ ID NO:62.

31. (canceled)

32. (canceled)
